(19) United States
(12) Patent Application Publication
(10) Pub. No.: US 2004/0259082 A1
(43) Pub. Date: Dec. 23, 2004

(54) Polymerases with charge-switch activity and methods of generating such polymers

(75) Inventor: John G. K. Williams, Lincoln, NE (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LI-COR, Inc., Lincoln, NE

(21) Appl. No.: 10/131,998

(22) Filed: Apr. 24, 2002

Related U.S. Application Data
(60) Provisional application No. 60/314,746, filed on Aug. 24, 2001.
(60) Provisional application No. 60/286,238, filed on Apr. 24, 2001.

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

This invention provides DNA polymerases with mutations in the charge-switch nucleotide interaction region that increase activity for charge-switch nucleotides. Such polymerases can be generated by introducing mutations in specific residues which are identified as being in the appropriate region through structural models, by homology to polymerases with known structures, or experimental analysis. In some embodiments, the mutant DNA polymerases have additional mutations that decrease activity for non-charge-switch nucleotides and mutations that decrease exonuclease activity. In another aspect, the invention provides methods of sequencing a target nucleic acid with the above described mutated DNA polymerases. In yet another aspect, the invention provides methods of generating polypeptides having charge-switch nucleotide polymerase activity by introducing “random” mutations and selecting those mutated polypeptides that encode polypeptides having charge-switch nucleotide activity

### CROSS-REFERENCES TO RELATED APPLICATIONS

This application claims priority to and incorporates by reference provisional applications U.S. Pat. App. No. 60/286,238, Attorney Docket No. 020031-001800US, filed Apr. 24, 2001, and U.S. Pat. App. No. 60/314,746, Attorney Docket No. 020031-001810US, filed Aug. 24, 2001. In addition, this application incorporates by reference the following related applications: PCT Pat. App. No. 2001/18699, Attorney Docket No. 020031-000810PC, filed Jun. 7, 2001; U.S. patent application Ser. No. 09/876,374, Attorney Docket No. 020031-000810US, filed Jun. 6, 2001; U.S. Pat. App. No. 60/340,522, Attorney Docket No. 020031-000811US, filed Dec. 12, 2001, and U.S. patent application Ser. No. 09/876,375, Attorney Docket No. 020031-000820US, filed Jun. 6, 2001.

### STATEMENT REGARDING GOVERNMENT RIGHTS TO THE INVENTION

This invention was made with Government support under the R44 HG02292 grant awarded by the PHS. The Government has certain rights to this invention.

### BACKGROUND OF THE INVENTION

The primary sequences of nucleic acids are crucial for understanding the function and control of genes and for applying many of the basic techniques of molecular biology. In fact, rapid DNA sequencing has taken on a more central role after the goal to elucidate the entire human genome has been achieved. DNA sequencing is an important tool in genomic analysis as well as other applications, such as genetic identification, forensic analysis, genetic counseling, medical diagnostics, and the like. With respect to the area of medical diagnostic sequencing, disorders, susceptibilities to disorders, and prognoses of disease conditions can be correlated with the presence of particular DNA sequences, or the degree of variation (or mutation) in DNA sequences, at one or more genetic loci. Examples of such phenomena include human leukocyte antigen (HLA) typing, cystic fibrosis, tumor progression and heterogeneity, p53 proto-oncogene mutations and ras proto-oncogene mutations (see, Gyllensten et al., PCR Methods and Applications, 1: 91-98 (1991); U.S. Pat. No. 5,578,443, issued to Santamaria et al.; and U.S. Pat. No. 5,776,677, issued to Tsui et al.).

Various approaches to DNA sequencing exist. The dideoxy chain termination method serves as the basis for all currently available automated DNA sequencing machines. (see, Sanger et al., Proc. Natl. Acad. Sci., 74: 5463-5467 (1977); Church et al., Science, 240: 185-188 (1988); and Hunkapiller et al., Science, 254: 59-67 (1991)). Other methods include the chemical degradation method, (see, Maxam et al., Proc. Natl. Acad. Sci., 74: 560-564 (1977), whole-genome approaches (see, Fleischmann et al., Science, 269, 496 (1995)), expressed sequence tag sequencing (see, Velculescu et al., Science, 270, (1995)), array methods based on sequencing by hybridization (see, Koster et al., Nature Biotechnology, 14, 1123 (1996)), and single molecule sequencing (SMS) (see, Jett et al., J. Biomol. Struct. Dyn. 7, 301 (1989) and Schecker et al., Proc. SPIE-Int. Soc. Opt. Eng. 2386, 4 (1995)).

PCT Application No. U.S. Ser. No. 99/29585, filed Dec. 13, 1999, and incorporated herein by reference, discloses a single molecule sequencing method on a solid support. The solid support is optionally housed in a flow chamber having an inlet and outlet to allow for renewal of reactants that flow past the immobilized polymerases. The flow chamber can be made of plastic or glass and should either be open or transparent in the plane viewed by the microscope or optical reader. Electro-osmotic flow requires a fixed charge on the solid support and a voltage gradient (current) passing between two electrodes placed at opposing ends of the solid support. The flow chamber can be divided into multiple channels for separate sequencing.

More recently, PCT application Ser. No. US00/13677, filed May 18, 2000, discloses a method of sequencing a target nucleic acid molecule having a plurality of bases. The temporal order of base additions during the polymerization reaction is measured on a molecule of nucleic acid. The activity of a nucleic acid polymerizing enzyme on the template nucleic acid molecule is thereafter followed in time. The sequence is deduced by identifying which base is being incorporated into the growing complementary strand of the target nucleic acid by the polymerizing enzyme at each step in the sequence of base additions. The steps of providing labeled nucleotide analogs, polymerizing the growing nucleic acid strand, and identifying the added nucleotide analog are repeated so that the nucleic acid strand is further extended and sequenced.

In addition, U.S. Pat. No. 4,979,824, illustrates that single molecule detection can be achieved using flow cytometry wherein flowing samples are passed through a focused laser with a spatial filter used to define a small volume. Moreover, U.S. Pat. No. 4,793,705 describes a detection system for identifying individual molecules in a flow train of the particles in a flow cell. The patent further describes methods of arranging a plurality of lasers, filters and detectors for detecting different fluorescent nucleic acid base-specific labels.

Single molecule detection on solid supports is described in Ishikawa, et al. Jan. J. Apple. Phys. 33:1571-1576. (1994). As described therein, single-molecule detection is accomplished by a laser-induced fluorescence technique with a position-sensitive photon-counting apparatus involving a photon-counting camera system attached to a fluorescence microscope. Laser-induced fluorescence detection of a single molecule in a capillary for detecting single molecules in a quartz capillary tube has also been described. The selection of lasers is dependent on the label and the quality of light required. Diode, helium neon, argon ion, argon-krypton mixed ion, and Nd:YAG lasers are useful in this invention (see, Lee et al. (1994) Anal Chem., 66:4142-4149).

Current high-throughput automated DNA sequencing is based on the pioneering methodology of Sanger et al. (1977) whereby labeled DNA elongation is randomly terminated within particular base groups through the incorporation of chain-terminating inhibitors (generally dideoxynucleoside triphosphates) and size-ordered by either slab gel electrophoresis or capillary electrophoresis. There have been several improvements in this automated technology since it was first reported in the mid-1980's with enhancements in the areas of separating technologies (both in hardware formats & electrophoresis media), fluorescence dye chemistry, polymerase engineering, and applications software. The emphasis on sequencing the human genome with a greatly accelerated timetable along with the introduction of capillary electrophoresis instrumentation that permitted more automation with respect to the fragment separation process allowed the required scale-up to occur without undue pressure to increase laboratory staffing. However, the reductions from such enhancements in the cost of delivering finished base sequence have been marginal, at best.

In general, present approaches to improve DNA sequencing technology appear to have taken one of two tacks:

1) continued emphasis to enhance throughput while reducing costs via the traditional Sanger methodology, such as increasing the number of capillary channels; miniaturization to permit microchannel separation with novel sample loading configurations and increased number of sample channels; and efforts to reduce the costs of Sanger fragment preparation through the use of greatly reduced sample volumes; and

2) paradigm shifts away from Sanger methodology such as sequencing by hybridization or the use of exonuclease to analyze base by base the terminus end of a DNA fragment.

U.S. Pat. No. 6,255,083 describes novel methods for target nucleic acid sequencing involving single molecule detection of fluorescently labeled PPi moieties released during synthesis of strands of nucleic acid complementary to the target nucleic acid. WO01/94609 describes modified nucleotides for use in such methods, wherein the nucleotide has a first molecular charge in the uncleaved form and a different molecule charge upon cleavage of the terminal phosphate. The “charge-switch” properties of these nucleotides allow separation of the cleaved terminal phosphate from the intact nucleotide phosphate probe reagents. This characteristic is useful for single-molecule DNA sequencing in a microchannel sorting system with an energy field. Using 4 different NTPs each labeled with a unique dye, real-time DNA sequencing is possible by detecting the released pyrophosphate having different labels. By electrically sorting differently charged molecules in this manner, the cleaved PPi-Dye molecules are detected in isolation without interference from unincoporated NTPs and without illuminating the polymerase-DNA complex.

φ29-type polymerases are valued for their strong strand displacement activity and ability to synthesize DNA strands several kilobases in length in rolling circle amplification. This makes them particularly attractive for use in many applications, including traditional sequencing methods.

Blanco et al. (U.S. Pat. No. 5,576,204) describe improved versions of 429-type polymerases with reduced exonuclease activity for use in traditional sequencing, but do not describe modification of other functional aspects of the enzyme.

Brandis et al. (U.S. Pat. No. 6,265,193) describe purified Taq DNA polymerases with specific mutations in the nucleobase interaction region that increase the incorporation of nucleotides labeled via the nucleoside base. Brandis et al. also describe polynucleotides encoding such polymerases, host cells, expression vectors, kits, and methods for using such polymerases in sequencing techniques. However, Brandis et al. do not describe any mutations in polymerase regions that interact with nucleotides labeled on the γ-phosphate, with charged moieties attached to the base, or labels attached to the sugar. Moreover, it is appreciated by those of skill in the art that the ability of certain mutations to influence Taq DNA polymerase activity with respect to labeled nucleotides cannot be extrapolated to other polymerases with low homology to Taq polymerases.

A need currently exists for more effective and efficient compounds, methods, and systems for charge-switch nucleotide sequencing. Specifically, a need exists for improved polymerases with properties optimized for use in charge-switch nucleotide sequencing, methods of using such polymerases, and methods of generating such polymerases. These and further needs are provided by the present invention.

### BRIEF SUMMARY OF THE INVENTION

In certain aspects, the invention provides purified DNA polymerases with mutant charge-switch nucleotide interaction pockets that optimize activity for charge-switch nucleotides, decrease activity for non-charge-switch nucleotides, and decrease exonuclease activity. While most naturally occuring polymerases have limited activity for charge-switch nucleotides, these purified DNA polymerases have considerably enhanced activity with respect to such nucleotides, making them particularly useful in single molecule sequencing methods.

In one aspect, the invention comprises a purified φ29-type DNA polymerase having at least one amino acid change as defined with respect to a naturally occurring φ29-type DNA polymerase, wherein the at least one amino acid change is in a charge-switch nucleotide interaction region and the DNA polymerase has increased activity for a charge-switch nucleotide. Typically, the mutations are either in the nucleotide γ-phosphate interaction region, the base interaction region, the sugar interaction region, or combinations thereof.

In a preferred embodiment, the mutation is in the nucleotide γ-phosphate interaction region, which comprises amino acids, including, but not limited to, Ile-115, His-116, Ile-179, Gln-180, Phe-181, Lys-182, Gln-183, Gly-184, Leu-185, Val-247, Phe-248, Asp-249, Val-250, Asn-251, Ser-252, Leu-253, Pro-255, Ala-256, Gly-350, Leu-351, Lys-352, Phe-353, Lys-354, Ala-355, Thr-356, Thr-357, Gly-358, Leu-359, Phe-360, Lys-361, Asp-362, Phe-363, Ile-364, Asp-365, Lys-366, Trp-367, Thr-368, Tyr-369, Ile-370, Lys-371, Thr-372, Thr-373, Ser-374, Glu-375, Gly-376, Ala-377, Ile-378, Lys-379, Gln-380, Leu-381, Ala-382, Lys-383, Leu-384, Met-385, Leu-386, Asn-387, Asp-458, Ser-459, Trp-483, Ala-484, His-485, Glu-486, Ser-487, Thr-488, Phe-489, Ile-501, Gln-502, Asp-503, Ile-504, Tyr-505, Met-506, Lys-507, Glu-508, Val-509, or Asp-510. In an especially preferred embodiment, the mutant DNA polymerase has a mutation of Lys-383, e.g., a K383A mutation.

In another embodiment, the mutation is in the base interaction region, preferably, at one of the following amino acid positions: Thr-117, Val-118, Ile-119, Tyr-120, Asp-121, Asp-200, Ile-201, Ile-202, Thr-203, Thr-204, Lys-205, Lys-206, Phe-207, Lys-208, Lys-209, Ala-225, Tyr-226, Arg-227, Gly-228, Gly-229, Phe-230, Thr-231, Trp-232, Leu-233, Asn-234, Asp-235, Arg-236, Ser-388, Leu-389, Tyr-390, Gly-391, Phe-393, Ala-394, Ser-395, Asn-396, Pro-397, Asp-398, Gln-497, Lys-498, Thr-499, Lys-512, Leu-513, Val-514, Glu-515, Gly-516, or Ser-517.

In yet another embodiment, the mutation is in the sugar interaction region, preferably, at either Tyr-254, Tyr-390, or Thr-457.

In an especially preferred embodiment, mutant DNA polymerases have decreased activity for a non-charge-switch nucleotide compared to the activity of a naturally occurring φ29-type DNA polymerase for a non-charge-switch nucleotide. The decrease can be about 20-fold.

In other embodiments, the mutant DNA polymerase has decreased exonuclease activity or completely lacks exonuclease activity. Preferably, it retains strand displacement activity. Mutations that reduce exonuclease activity and retain strand displacement activity include mutations of Asn-62 or Thr-15, e.g., N62D or T15I mutations.

The mutant DNA polymerases of this invention can have multiple mutations. In especially preferred embodiments, the mutant φ29-type DNA polymerases have one of the following sequences: SEQ ID NOs:4-36.

The mutant φ29-type polymerases of this invention can come from phages including, but not limited to, φ29, Cp-1, PRD1, φ15, φ21, PZE, PZA, Nf, M2Y, B103, SF5, GA-1, Cp-5, Cp-7, PR4, PR5, PR722, and L17. Preferably, the 429-type polymerase is a DNA polymerase from a φ29 phage.

In another aspect, the invention comprises a method for sequencing a target nucleic acid with a purified φ29-type DNA polymerase. The method comprises:

a) immobilizing a complex comprising the purified φ29-type DNA polymerase or a target nucleic acid onto a solid phase in a single molecule configuration, wherein the purified φ29-type DNA polymerase has at least one amino acid change as defined with respect to a naturally occurring φ29-type DNA polymerase, wherein the at least one amino acid change is in the charge-switch interaction region, the purified φ29-type DNA polymerase having increased activity for a charge-switch nucleotide;

b) contacting the complex with a primer nucleic acid which complements a region of the target nucleic acid of the region to be sequenced and a sample stream comprising a target nucleic acid when the purified DNA polymerase is immobilized or the purified DNA polymerase when the target nucleic acid is immobilized and a charge-switch nucleotide having a detectable moiety, wherein the detectable moiety is released as a charged detectable moiety when the charge-switch nucleotide is incorporated into the primer nucleic acid wherein the solid support is attached to a flowcell having an inlet port and an outlet port;

c) applying an energy field to the sample stream; and

d) detecting the charged detectable moiety, thereby sequencing the target nucleic acid.

In yet another aspect, the invention comprises a method for generating a polypeptide having charge-switch nucleotide polymerase activity, the method comprising:

(a) providing a parent polynucleotide;

(b) mutating the polynucleotide to generate a library of mutated polynucleotides; and

(c) selecting from the library a mutated polynucleotide encoding a polypeptide having charge-switch nucleotide polymerase activity. In certain embodiments, the step of selecting a mutated polypeptide further comprises selecting a polypeptide with reduced non-charge-switch nucleotide polymerase activity and decreased exonuclease activity. In some embodiments, the mutated polynucleotide is selected via PCR.

In certain embodiments, the parent polynucleotide encodes an active φ29-type polymerase. The parent polynucleotide can also encode other polymerases including, but not limited to, HIV reverse transcriptase or a T7 polymerase. In preferred embodiments, the parent polynucleotide used in the method for generating an improved polymerase encodes an inactive φ29-type polymerase. In especially preferred embodiments, the parent polynucleotide has been further mutated to eliminate exonuclease activity.

The step of mutating the parent polynucleotide can comprise methods including, but not limited to, in vitro recombination, in vivo recombination, single-site or multi-site directed mutagenesis, error-prone PCR mutagenesis, and site-saturation mutagenesis. In some embodiments, the method further comprises: (d) shuffling of at least two mutated polynucleotides and (e) selecting another mutated polynucleotide encoding a polypeptide having charge-switch nucleotide polymerase activity. Alternatively, the method comprises (d) shuffling of a mutated polynucleotide and a polynucleotide encoding a different polymerase with sufficient nucleotide homology to permit shuffling; and (e) selecting another mutated polynucleotide encoding a polypeptide having charge-switch nucleotide polymerase activity.

These and other objects and advantages will become more apparent when read with the accompanying detailed description and drawings that follow.

### BRIEF DESCRIPTION OF THE DRAWINGS

FIG. 1 illustrates an approach to single-molecule sequencing that utilizes charge switching to separate PPi-F groups from excess γ-dNTPs in a microfluidics sorting system. Intact nucleotides flow in a microchannel from the bottom of the figure toward a single immobilized polymerase-DNA complex (bead). Upon incorporation into DNA, the dye is cleaved from the nucleotide along with pyrophosphate to acquire a net positive charge; an electric field forces the PPi-F into the right-side channel where it is detected with single-molecule sensitivity.

FIG. 2 illustrates a computer model of a microfluidics embodiment of the present invention.

FIG. 3 illustrates a bead trap embodiment of the sequencing method of this invention. Three frames of a movie demonstrate bead trapping by “suction” at a small wall-port in a microchannel 12 μm wide×6 μm deep. Frame 1 A string of 4 μm beads (1) is retained momentarily under suction at a constricted 2 μm port in the channel wall (2). Frame 2 The string breaks free (3), leaving a single bead (4) behind. Frame 3 The single bead (4) is retained for the duration of the movie.

FIG. 4 illustrates the probability of detecting a single molecule as a function of the photophysics of the particular dye. Panel B, dashed vertical line (at arrows) is the detection threshold of 60 photons.

FIG. 5 illustrates one embodiment of overall sequencing error as a function of individual base detection efficiency and oversampling factor, assuming a requirement of at least 33% hits in a sampling ensemble.

FIG. 6 illustrates the utilization of different γ-dNTPs by T7 Sequenase 2.0 and HIV polymerases. Samples contain 50 μM dATP, dCTP, dGTP and either (a) dUTP; (b) γ-dUTP-BodipyTR; (c) γ-dUTP-Fluorescein; or a control (d) omitting dUTP and its analogs. Incubation was at 37° C. for 30 min. Bracket indicates stopped synthesis at run of 7 dUTP incorporation sites in the primed template.

FIG. 7 illustrates the expression of φ29 HP-thio polymerase.

FIG. 8 show the expression (A) and purification (B) of T7 DNA polymerase. Panel C shows a Western blot analysis of protein purified in 96-well format. Soluble protein from induced and uninduced cultures was probed with anti-XPress antibody (Invitrogen), which recognizes an XPress epitope fused to the N-terminus of the polymerase.

FIG. 9 illustrates the Kₘ determination for dTTP. Samples (10 μL) contained 40 mM TrisCl pH7.5, 10 mM MgCT₂, 50 mM NaCl, 100 ug/ml BSA, 300 μM each of dATP, dCTP, dGTP, and dTTP from 0 to 35 μM (lanes 1-9), 50 nM template, 25 nM IRD-labeled primer, 50 nM T7 polymerase exo-. Polymerase was pre-incubated for 5 min on ice with 1000-fold excess E. coli thioredoxin that contained 5 mM DTT. Incubation was for 5 sec at 20° C. and the reaction was quenched. Primer extension products were analyzed on a fluorescence sequencer. Fraction of primer converted to full-length extension product is graphed in a Lineweaver-Burk plot.

FIG. 10 illustrates an assay for polymerase activity based on the high specificity of UDG for uracil-containing DNA. (A) Assay scheme, (B) Demonstration using a uracil-containing 100-mer template “U-DNA”, test-primer, and a second PCR primer (5′-ACCTTTGACGTGGCGTG). Double-stranded “T-DNA” was prepared in advance by primer extension using dNTPs containing dTTP and Taq polymerase at 72° C. for 5 min. Test samples (10 μL) contained 5E10 molecules of primed U-DNA, plus SE06, SE05, 5E04 or 0 molecules of D-DNA (lanes 1-4, respectively, indicated by the ratio of D-DNA to U-DNA) in 50 mM TrisCl pH 9, 20 mM NaCl, UDG (100 u/ml; Epicentre H-UNG). After incubating at 44° C. for 60 min, samples were heated at 95° C. to inactivate the UDG and to cleave abasic sites in the treated DNA. Two μL of each sample was diluted into a final volume of 10 UI containing 1× TaqGold Master Mix (Applera), 2.5 mM MgCl₂, 200 μM each dATP, dCTP, dGTP, dUTP, 1 μM each of the first and second PCR primer (above) and TaqGold polymerase (100u/ml). The PCR conditions were 95° C. 10 min, 35× (94° C. 45s, 60° C. 45s, 72° C. 45s) 72° C. 5 min, 4° C. hold. Electrophoresis was in a 4% E-Gel (Invitrogen).

FIG. 11 illustrates the lack of polymerase activity of the T7 pol-mutant. The T7 pol-mutant was tested for activity using the primer extension assay of FIG. 9. (Lane 1) Pol+control, 4 dNTPs. (Lane 2) Pol+control, dTTP only. (Lane 3) Pol+control, no dNTPs. (Lane 4) complete reaction with pol-mutant.

FIG. 12 illustrates the equilibrium calculations showing the effect of Mg⁺⁺ on the time-averaged electric charge on the “ligands” N-PPP—F and PP—F. The fraction of ligand bound to an ion, fracBound, is given as fracBound =([ion]/([ion] +K), where K is the ion concentration giving fracbound=50% (i.e., the association or dissociation constant). Values for K are extrapolated from the various characterized nucleotides and phosphate compounds. K for Mg/PP—F is taken from ADP, CDP and PP—H (log(K)=3.21, 3.22, 3.18 respectively). The protonated forms (secondary ionization) ATP—H and CTP—H (log(K)=2.18 and 2.18) are models. The protonated forms (secondary ionization) ADP—H and CDP—H (log(K)=1.55 and 1.60) are models for H—PP—F. The primary ionizations are log(K)-2 for all compounds. The phosphate secondary ionizations average at log(K)=6.55 (average of 6.41, 6.47, 6.38, 6.40, 6.57, 6.59, 6.63 for ADP, CDP, ATP and CTP).

FIG. 13 illustrates the effect of Mg++on electrophoretic migration of the γ-dNTP (Panel A) in agarose gels containing the indicated amounts of Mg⁺⁺.

FIG. 14 illustrates the effect of Mg⁺⁺ on electrophoretic mobility of unlabeled nucleotides.

FIG. 15 illustrates efficient utilization of γ-dTTP (++)-BTR by T7 DNA polymerase exo-. Samples contained 50 mM IRD700-labeled primer, 100 nM template, 100 nM polymerase, 20 μM each dNTP with either unlabeled or γ-labeled dTTP. Incubation times (a-f) were 5, 10, 30, 60, 90 and 120 sec at 20° C.

FIG. 16 illustrates that there is no dTTP contamination in other components of the reaction mix. Lane 1 is a negative control showing the primed single-strand template. Lanes 2 and 4 show the fully-double-stranded primer extension product made with unlabeled dTTP. Lane 5 shows the same product made with γ-dTTP-BQS434-BodipyTR. Lane 7 shows that no product is made when dTTP and the γ-dTTP are omitted from the otherwise-complete reaction mix, establishing that there is no dTTP contamination in any of the other components. Lane 8 and 9 show that neither γ-dTTP nor dTTP are contaminated with A+C+G.

FIG. 17 illustrates that aminoallyl(+)dUTP is utilized by T7 Sequenase 2.0 and HIV-RT, but not by Klenow or Taq. Samples contain dATP, dCTP, dGTP and either dUTP (first lane of each enzyme) or AA-dUTP (second lane each enzyme). Arrows indicate the extension products. Incorporation of AA-dUTP gives a product having slower electrophoretic mobility than incorporation of unlabeled dUTP.

FIG. 18 illustrates one embodiment of a flowchart of the breeding process.

FIG. 19 illustrates different schemes for synthesizing various types of γ-dNTPs.

FIG. 20 illustrates additional schemes for synthesizing various types of γ-dNTPs.

FIG. 21 illustrates the method used for isolation of clones with the desired activity.

FIG. 22 illustrates an electrophoretic gel in one embodiment of the present invention. R518 coordinates a γ-P oxygen; D654 coordinates an active-site Mg⁺⁺ 1=5 sec reaction; 2=30 sec; 3=300 sec; N=no enzyme control The reaction conditions are as follows: 50 nM template (50 bp “mid-7”), 50 nM IR700 M13 primer, 20 uM each dNTP, 100 nM “WT” polymerase that is an exonuclease deficient mutant.

FIG. 23 illustrates a structural model of the φ29 polymerase complexed with a γ-dNTP. Amino acids comprising the γ-P pocket are in white. The γ-dNTP is enclosed by the circle. The linker attached to the γ-P is the thick line. The detectable tag is “F”.

FIG. 24 illustrates single molecule sequencing by electrosorting. As shown herein, the target DNA strand is immobilized on a bead trapped in a microchannel. Pressure-driven flow moves polymerase and all 4 charge-switch dNTPs past the DNA as indicated (vertical arrow). Nucleotide incorporation generates labeled pyrophosphate PPi-F. In the example shown, the dNTP is negative and the PPi-F is positive. An electric field in the horizontal channel drives intact dNTPs to the left and PPi-F to the right where it is detected by fluorescence.

FIG. 25 illustrates a charge-switch dUTP. As shown, the dye has a net charge of zero (zwitterionic +1/−1), the linker has two quaternary amines that contribute a charge of (+2), and the base has a carboxylate group having a charge of (−1).

FIG. 26 illustrates a charge-switch dUTP and PPi-F being sorted in opposite directions. The two components were introduced by pressure-driven bulk flow into a microfluidics cross at opposite ports. The intact nucleotide (more negative) moved from the left port toward the positive electrode, while the PPi-F (less negative) moved the opposite way.

FIG. 27 illustrates the expression and purification of His-tagged φ29 DNA polymerase wherein protein expression is induced by arabinose and samples were processed as described. PAGE-SDS gel: insoluble fraction (lane 1), soluble fraction (lane 2), purified protein (lane 3). Full-length φ29-HisTag protein is marked by the arrow.

FIG. 28 illustrates strand-displacement activity of his-tagged φ29 DNA polymerase. Primer extension on a single-stranded M13 DNA template. Size standard (Stratagene “kb ladder”; lane 1), control M13 DNA without polymerase (lane 2), plus φ29 polymerase (lane 3), plus Klenow DNA polymerase (lane 4). Strand-displacement synthesis by phi29 polymerase is evident by production of M13 concatemers too large to enter the gel (arrow, lane 3). Klenow polymerase was relatively incapable of strand-displacement synthesis (lane 4).

FIG. 29 illustrates positions of N62D and K383A mutations in φ29 DNA polymerase. The nucleotide (N), N62D (Exo) and K383A (Pol) mutations are mapped in a structural model of φ29 polymerase built based on sequence homology to polymerases of known structure.

FIG. 30 illustrates a screening assay based on the high specificity of UDG for uracil-containing DNA. (A) Assay scheme. (B) Demonstration using a uracil-containing 100-mer template.

### DETAILED DESCRIPTION OF THE INVENTION AND PREFERRED EMBODIMENTS

I. Definitions

The term “amino acid change” as used herein, refers to any mutation where the amino acid residue at a particular position in a sequence is different from that found at the corresponding location in the naturally occurring sequence. Such mutations can be conservative changes or non-conservative changes.

The term “non-conservative mutation” or “non-conservative change” as used herein applies to both amino acid and nucleic acid sequences. With respect to particular nucleic acid sequences, “non-conservative mutations” refers to those nucleic acid changes which do not encode identical or essentially identical amino acid sequences, or where the nucleic acid does not encode an amino acid sequence, to sequences which have different nucleotide sequences.

As to amino acid sequences, one of skill will recognize that individual substitutions, deletions or additions to a nucleic acid, peptide, polypeptide, or protein sequence which alter, add or delete a single amino acid or a small percentage of amino acids in the encoded sequence is a “non-conservative mutation” where the alteration results in the substitution of an amino acid with a chemically dissimilar amino acid.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “conservative or silent variations”. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

Conservative substitution tables providing functionally similar amino acids are well known in the art. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The term “charge-switch nucleotide”, “NP probe”, or “γ-dNTP” as used herein refers to a phosphate-labeled nucleotide (e.g., γ-NP-Dye) that upon release or cleavage of a detectable moiety (e.g., PPi-Dye) has a different net charge associated with the cleavage product compared to the intact nucleotide probe (e.g., γ-NP-Dye). In certain preferred aspects, the attachment of the dye to the PPi is via a nitrogen in lieu of an oxygen. Preferably, the charge difference between the intact labeled nucleotide and the cleavage product is at least 0.5, and more preferably about 1 to about 4 (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0). In certain embodiments, the “charge-switch nucleotide” also has additional charged moiety on the base.

The term “non-charge-switch nucleotide” as used herein refers to any nucleotide which lacks a detectable phosphate moiety. For example, both naturally occurring dNTPs and dNTPs labeled solely on a base are considered to be “non-charge-switch nucleotides”.

The term “charge-switch nucleotide interaction region” as used herein refers to the portion of a DNA polymerase which binds, interacts with, or is in close proximity to charge-switch nucleotide triphosphates as they are incorporated into a newly synthesized strand of DNA.

The term “base interaction region” as used herein refers to the portion of a DNA polymerase which binds, interacts with, or is in close proximity to the base of nucleotide triphosphates as they are incorporated into a newly synthesized strand of DNA.

The term “sugar interaction region” as used herein refers to the portion of a DNA polymerase which binds, interacts with, or is in close proximity to the sugar of nucleotide triphosphates as they are incorporated into a newly synthesized strand of DNA.

The term “nucleotide γ-phosphate interaction region” as used herein refers to the portion of the DNA polymerase which binds, interacts with, or is in close proximity to the γ-phosphate and/or the linker fluorophore portion of the nucleotide triphosphates as they are incorporated into a newly synthesized strand of DNA.

The term “increased activity” as used herein refers to the enhanced ability of a DNA polymerase to bind and use nucleotides with certain properties as substrates for DNA synthesis. Such activity is preferably increased by at least 2-fold.

The term “decreased activity” as used herein refers to the decreased ability of a DNA polymerase to bind and use nucleotides with certain properties as substrates for DNA synthesis. Such activity is preferably decreased by 2-fold to 20-fold; more preferably, by 10-fold to 20-fold; and most preferably, by greater than 20-fold.

The term “φ29-type polymerase” refers to any DNA polymerase isolated from the related phages which contain a terminal protein used in the initiation of replication of DNA. These phages are generally described by Salas, 1 The Bacteriophages 169, 1988. The φ29-type polymerases include those polymerases from Cp-1, PRD1, φ15, φ21, PZE, PZA, Nf, M2Y, B103, SFS, GA-1, Cp-5, Cp-7, PR4, PRS, PR722, and L17 phages.

The term “inactive φ29-type polymerase” as used herein refers to a polymerase that has been mutated such that it is no longer capable of synthesizing DNA strands from either dNTPs or charge-switch nucleotides.

Positions of amino acid residues within a DNA polymerase are indicated by either numbers or number/letter combinations. The numbering starts at the amino terminus residue. The letter is the single letter amino acid code for the amino acid residue at the indicated position in the naturally occurring enzyme from which the mutant is derived. Unless specifically indicated otherwise, an amino acid residue position designation should be construed as referring to the analogous position in all DNA polymerases, even though the single letter amino acid code specifically relates to the amino acid residue at the indicated position in the φ29 polymerase (SEQ ID NO:1).

As used herein, the term “DNA shuffling”, “gene shuffling”, or “shuffling of DNA” is used herein to indicate recombination between substantially homologous but nonidentical sequences; in certain instances, DNA shuffling may involve crossover via nonhomologous recombination, such as via cre/lox and/or flp/frt systems and the like, such that recombination need not require substantially homologous polynucleotide sequences. By generating molecular chimeras and/or molecular hybrids of substantially dissimilar sequences, DNA shuffling allows for accelerated and directed protein evolution in vitro. See, U.S. Pat. No. 6,117,679, issued to Stemmer on Sep. 12, 2000, which is incorporated herein by reference.

The terms “PPi-Dye” or “PP—F” and the like, refer to the pyrophosphate cleavage product from an intact charge-switch nucleotide (NTP). If a nucleotide diphosphate is used, the cleavage product will be a “P-Dye” or “P—F”.

The phrase “phosphate detectable moiety” refers to a detectable cleavage product from a NP probe of the present invention. Examples include, but are not limited to, PPi-Dye, PP—F, P-Dye, a phosphate fluorophore moiety, a terminal phosphate fluorophore moiety, a detectable moiety, charged groups, electrically active groups, detectable groups, reporter groups, combinations thereof, and the like.

The term “oligonucleotide” as used herein includes linear oligomers of nucleotides or analogs thereof, including deoxyribonucleosides, ribonucleosides, and the like. Usually, oligonucleotides range in size from a few monomeric units, e.g. 3-4, to several hundreds of monomeric units. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, and “T” denotes thymidine, unless otherwise noted.

The term “nucleoside” as used herein refers to a compound consisting of a purine, deazapurine, or pyrimidine nucleoside base, e.g., adenine, guanine, cytosine, uracil, thymine, deazaadenine, deazaguanosine, and the like, linked to a pentose at the 1′ position, including 2′-deoxy and 2′-hydroxyl forms, e.g., as described in Kornberg and Baker, DNA Replication, 2nd Ed. (Freeman, San Francisco, 1992).

The term “nucleotide” as used herein refers to a phosphate ester of a nucleoside, e.g., mono, di and triphosphate esters, wherein the most common site of esterification is the hydroxyl group attached to the C-5 position of the pentose. Nucleosides also include, but are not limited to, synthetic nucleosides having modified base moieties and/or modified sugar moieties, e.g. described generally by Scheit, Nucleotide Analogs (John Wiley, N.Y., 1980). Preferably, the modified nucleotide triphosphates used in the methods of the present invention are selected from the group of dATP, dCTP, dGTP, dTTP, dUTP and mixtures thereof.

The term “primer” refers to a linear oligonucleotide, which specifically anneals to a unique polynucleotide sequence and allows for synthesis of the complement of the polynucleotide sequence. In certain aspects, a primer is covalently attached to the template as a hairpin.

The phrase “sequence determination” or “determining a nucleotide sequence” in reference to polynucleotides includes determination of partial as well as full sequence information of the polynucleotide. That is, the term includes sequence comparisons, fingerprinting, and like levels of information about a target polynucleotide, or oligonucleotide, as well as the express identification and ordering of nucleosides, usually each nucleoside, in a target polynucleotide. The term also includes the determination of the identification, ordering, and locations of one, two, or three of the four types of nucleotides within a target polynucleotide.

The term “heterogeneous” assay as used herein refers to an assay method wherein at least one of the reactants in the assay mixture is attached to a solid phase, such as a solid support.

The term “solid phase” refers to a material in the solid phase that interacts with reagents in the liquid phase by heterogeneous reactions. Solid phases can be derivatized with proteins such as enzymes, peptides, oligonucleotides and polynucleotides by covalent or non-covalent bonding through one or more attachment sites, thereby “immobilizing” the protein or nucleic acid to the solid phase, e.g., solid-support.

The phrase “target nucleic acid” or “target polynucleotide” refers to a nucleic acid or polynucleotide whose sequence identity or ordering or location of nucleosides is to be determined using methods described herein.

The phrase “terminal phosphate oxygen” refers to the secondary ionization oxygen atom (pK ˜6.5) attached to the terminal phosphate atom in a nucleotide phosphate probe.

The phrase “internal phosphate oxygen” refers to the primary ionization oxygen atoms (pK ˜2) in a nucleotide phosphate probe. An NTP has 3 internal phosphate oxygens (one each on the α, β, and γ-phosphates) plus 1 terminal phosphate oxygen (on the γ-phosphate).

The phrase “single molecule configuration” refers to the ability of the compounds, methods and systems of the present invention to measure single molecular events, such as an array of molecules on a solid support wherein members of the array can be resolved as individual molecules located in a defined location. The members can be the same or different.

II. Overview

This invention provides DNA polymerases with mutations in the charge-switch nucleotide interaction region that increase polymerase activity for charge-switch nucleotides. Such polymerases can be generated by introducing mutations in specific residues which are identified as being in the appropriate region through structural models, by homology to polymerases with known structures, or by experimental characterization (e.g., site-directed mutagenesis). In some cases, the DNA polymerase has additional mutations that decrease activity for non-charge-switch nucleotides and mutations that decrease exonuclease activity. Preferably, the mutant polymerase is capable of synthesizing DNA at a rate of at least 1 nt/sec; more preferably, at least 10 nts/sec; most preferably, at least 100 nts/sec.

In another aspect, the invention provides methods of sequencing a target nucleic acid with the above described mutated DNA polymerases.

In yet another aspect, the invention provides methods of generating polypeptides having charge-switch nucleotide polymerase activity by introducing “random” mutations and selecting those mutated polypeptides that encode polypeptides having charge-switch nucleotide activity. In certain embodiments, the invention also provides mutant polymerases identified by such methods.

III. Charge-Switch Nucleotides

As described, the polymerases of the present invention possess activity for charge-switch nucleotides (“NP probes”) as substrates. The methods for making, using and multiple examples of charge-switch nucleotides are described in detail in International Publication No. WO 01/94609, published to Williams et al, on Dec. 13, 2001, which is incorporated herein by reference. Further charge-switch nucleotides are described in U.S. patent application Ser. Nos. 09/879,374 and 09/879,375, filed on Jun. 6, 2001, as well as U.S. Provisional Application No. 60/340,522, filed Dec. 12, 2001, and entitled, “Charge-Switch Nucleotides.” The foregoing applications are incorporated herein by reference.

In general, the term “charge-switch nucleotide” refers to a labeled intact nucleotide phosphate (e.g., γ-NP-Dye) whereupon release or cleavage of a phosphate detectable moiety (e.g., PPi-Dye) using for example, a polymerase of the present invention, has a different net charge associated with the cleavage product compared to the intact nucleotide phosphate probe (e.g., γ-NP-Dye). In certain preferred aspects, the attachment of the dye to the PPi is via a nitrogen in lieu of an oxygen. Preferably, the charge difference between the intact γ-NP-Dye and the PPi-Dye is at least 0.5, and more preferably about 1 to about 4.

As used herein, the phrase “phosphate detectable moiety” refers to a detectable cleavage product from a NP probe, e.g., “PPi-Dye”, “PP—F” and the like, or if a nucleotide diphosphate NP probe is used, the cleavage product will be a “P-Dye” or “P—F”. In certain embodiments, the polymerases of the present invention can be used to incorporate an NP probe into a growing complementary strand of nucleic acid. This reaction results in the release of a phosphate detectable moiety. The phosphate detectable moiety is preferably a γ-phosphate label that is cleaved from γ-labeled dNTPs. In one embodiment, γ-labeled-dNTPs having a cationic γ-label exhibit charge-switching behavior, wherein the electric charge of the intact triphosphate (γ-NTP-Dye) is negative while the released PPi-Dye is positive. Thus, the release of the PPi-Dye results in a cleavage-dependent charge alteration to the PPi-fluorophore moiety. In certain aspects, cleavage of pyrophosphate from the nucleoside subtracts charges associated with the nucleoside. These charge changes can be either positive or negative. In certain aspects, the cleavage of the PPi-Dye adds a positive charge to the PPi-Dye moiety by generating a terminal phosphate oxygen, as a terminal phosphate-oxygen binds mono or divalent cations (e.g., Mg⁺⁺, Mn⁺⁺, K⁺, Na⁺ and the like) as counter ions, better than an internal phosphate-oxygen.

In certain aspects, the intact charge-switch NP probes useful in the present invention have a net positive charge. For example, the base can have an amine attached thereto and this amine can be protonated. Upon cleavage of the base-cation, the PPi-Dye becomes more negative. Conversely, cleavage of a negative-base NP (e.g., a base with a carboxylate, sulfonate, and the like attached thereto) makes the PPi-Dye more positively charged. Cleavage of a neutral-base NTP (natural structure), will have no contribution to the PPi-Dye other than generation of a terminal phosphate oxygen.

In certain aspects, a charge-switch nucleotide comprises an intact NP probe having a terminal phosphate with a fluorophore moiety attached thereto. The intact NP probe has a first molecular charge associated therewith; and whereupon cleavage of the terminal phosphate such as cleavage of a pyrophosphate fluorophore moiety, the pyrophosphate fluorophore moiety carries a second molecular charge. The first molecular charge is different than the second molecular charge by at least 0.4 as calculated under ionic conditions obtained in pure water, at about pH 7. The charge difference between the intact NP probe is more preferably between about 1 and about 4, and any fraction of the integers 1, 2, and 3

The charge state of the either the γ-NP-Dye or terminal phosphate-Dye (e.g., PPi-Dye) or both can be determined for any ionic condition by calculating the i) charge on the base; ii) the charge on the fluorophore or linker; and iii) the buffer cation composition and concentration.

In general, the net electric charge on a nucleotide phosphate such as a dNTP, is governed by the base ring nitrogens and by the three phosphates. At a pH from about 6.5 to about 8.5, the bases are mostly uncharged (nitrogen pK of 3-4 and 9.5-10). The primary ionization of each ionizable oxygen atom on each phosphate (pK ˜2) contributes one full negative charge. The secondary ionization specific to the phosphate oxygen (pK˜6.5) contributes a time-averaged charge of −0.9 at pH 7.5 so the total charge on the dNTP is −3.9.

In certain aspects, the nucleobase carries a cationic adduct and the terminal oxygen is replaced by a nitrogen and a label moiety in a γ-dNTP, thus, the secondary ionization is eliminated and at pH 7 (H₂O), the charge on a γ-dNTP is −2.0 (for a neutral γ-label). After cleavage from the nucleotide, the charge on the PPi-Dye is −2.74, because it has lost the positive charge (+1) of the nucleobase, but has gained back a partial positive charge (+0.26) due to hydrogen ion equilibration with the terminal phosphate oxygen (pK 6.4 secondary ionization of substituted diphosphates).

The magnitude of a charge-switch nucleotide (“NP probe”) can be enhanced by attaching positive or negative charged groups to the nucleoside (normally neutral at pH 7.5). The range of the charge-switch can be set by attaching charged groups to the γ-phosphate label, either on the fluorophore and/or linker, such that both the NP probe and the PPi-F are negatively charged, or both are positively charged, or one is negative while the other is positive. All such combinations and permutations are useful in the present invention. Thereafter, when the base is incorporated into DNA, the charged group is separated from the PPi-F to enhance the “natural” counter ion (e.g., Mg⁺⁺) dependent charge effect.

In certain aspects, the charge difference between the intact NP probes and the detectable moieties can be introduced via a charged moiety fixed to the γ-label such that, the γ-NTP-Dye is net negative, while the PPi-Dye is net positive. For example, when the electroneutral dye TAMRA is conjugated to dTTP using a linker having a charge of +2 the γ-NTP-Dye is net negative, while the PPi-Dye is net positive in the presence of Mg++ ion. This nucleotide can be incorporated into DNA by using a polymerase of the present invention, with the release of phosphate, thus the PPi-Linker-Dye moiety acquires a more positive charge than the intact γ-NTP-Dye.

In certain aspects, charge-switch nucleotides of Formula I are useful for the polymerases of present invention. In this aspect, the NP probe has a terminal phosphate with a fluorophore moiety attached thereto, wherein the intact NP probe has a first molecular charge associated therewith, and upon cleavage of the fluorophore moiety having a phosphate or pyrophosphate group appended thereto, the P—F or PPi-F has a second charge. The first charge and second charge are different. Formula I provides charge-switch nucleotide phosphate probes of the present invention:

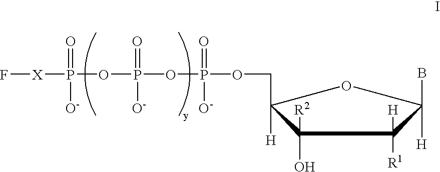

In Formula I, B is a nucleobase including, but not limited to, naturally occurring or synthetic purine or pyrimidine heterocyclic bases, including but not limited to adenine, guanine, cytosine, thymine, uracil, 5-methylcytosine, hypoxanthine or 2-aminoadenine. Other such heterocyclic bases include 2-methylpurine, 2,6-diaminopurine, 6-mercaptopurine, 2,6-dimercaptopurine, 2-amino-6-mercaptopurine, 5-methylcytosine, 4-amino-2-mercaptopyrimidine, 2,4-dimercaptopyrimidine and 5-fluorocytosine. Representative heterocyclic bases are disclosed in U.S. Pat. No. 3,687,808 (Merigan, et al.), which is incorporated herein by reference.

In certain aspects, B comprises a charged moiety. These charged base-moieties can be positively or negatively charged. Using a charged base-moiety, it is possible to impart additional charge onto the base or the intact γ-dNTP—F. Suitable charged base linking groups can append carboxylic acid group, sulfonic acid group, and the like.

R¹ in Formula I is a hydrogen, a hydroxyl group or charged group e.g., L-SO₃⁻, L-NH₃⁺, L-CO₂⁻ and the like; wherein L is a linker.

R² in Formula I is a hydrogen, or charged group e.g., L-SO₃⁻, L-NH₃⁺, L-CO₂⁻ and the like; wherein L is a linker.

In Formula I, X is a heteroatom such as nitrogen, oxygen, and sulfur. Preferably, X is nitrogen. As the NP probes of the present invention can be tetraphosphates, triphosphates or diphosphates, the index “y” in Formula I, can be 0, 1 or 3.

In Formula I, F is a fluorophore or dye. In certain preferred aspects, F comprises a charged label linker group. Using the charged label linking group, it possible to impart additional charge onto the fluorophore moiety (i.e., the cleaved PPi-F or P—F). Alternatively, F is appended to the terminal phosphate by a linker group, described in detail below. Suitable charged label-linking groups can append quaternary nitrogens and the like. The compounds of Formula I can have counter ions associated therewith. These counter ions include mono and divalent metal ions including, but are not limited to, Mg⁺⁺, Mn⁺⁺, K⁺ and Na⁺.

In certain aspects, the intact charge-switch nucleotide phosphate (NP) probes useful in the present invention have a functionalized sugar, whereupon enzymatic cleavage of the intact charge-switch NP probe, a detectable moiety is produced that migrates to an electrode, whereas the intact charge-switch NP probe migrates to the other electrode. In certain aspects, the sugar label can be cleaved from the NP probe either during incorporation, or after the nucleotide is incorporated. In the latter case, the detectable moiety (DM) on the sugar is actually incorporated into the DNA. The DM at the 3′-end of the DNA is released during incorporation of the next nucleotide. For example, a polymerases of the present invention will cleave a 3′-sugar label from the end of the primer when adding the next nucleotide to the primer.

In one aspect, the functionalized sugar can have the charged group(s) at C-2′, C-3′ or combinations thereof. Suitable charged groups and their syntheses are disclosed in U.S. Pat. No. 6,191,266 (incorporated herein by reference).

The functional group of the functionalized sugar can carry a positive charge or a negative charge. In one preferred embodiment, the intact charge-switch NP probe useful in the present invention is a compound of the formula:

NL₁L₂-DM II

wherein:

N is a nucleotide;

L₁L₂-DM is a functional group;

L₁ is a cleavable linking group, wherein one end of the cleavable linking group is attached to the 3′ position of the nucleotide;

L₂ is a spacer linking group; and

DM is a detectable moiety.

In certain preferred aspects, L₁ is selected from the group of NHC(O)—, NHC(S)—, CH₂C(O)—, OC(O)—, and OPO₃— and L₂ is selected from the group of —(NHCO)ₙ and —(OCH₂CH₂)ₙ. Preferably, the detectable moiety is a fluorophore.

In certain aspects, the intact charge-switch NP probe of the present invention have at least one member of L₁, L₂ and DM carrying at least one positive charge. Preferably, L₁ is selected from NHC(O)—, NHC(S)—, CH₂C(O)—, OC(O)—, and OPO₃—. L₂ is preferably selected from —(NHCO)ₙ and —(OCH₂CH₂)ₙ.

A. Labels

Many dyes or labels are suitable for charge-switch nucleotide phosphates of the present invention. In certain preferred aspects, suitable dyes include, but are not limited to, coumarin dyes, xanthene dyes, resorufins, cyanine dyes, difluoroboradiazaindacene dyes (BODIPY), ALEXA dyes, indoles, bimanes, isoindoles, dansyl dyes, naphthalimides, phthalimides, xanthenes, lanthanide dyes, rhodamines and fluoresceins. In certain embodiments, certain visible and near IR dyes are known to be sufficiently fluorescent and photostable to be detected as single molecules. In this aspect the visible dye, BODIPY R6G (525/545), and a larger dye, LI-COR's near-infrared dye, IRD-38 (780/810) can be detected with single-molecule sensitivity and are used to practice the present invention. In certain preferred aspects, suitable dyes include, but are not limited to, fluorescein, 5-carboxyfluorescein (FAM), rhodamine, 5-(2′-aminoethyl) aminonapthalene-1-sulfonic acid (EDANS), anthranilamide, coumarin, terbium chelate derivatives, Reactive Red 4, BODIPY dyes and cyanine dyes.

B. Linkers to the Label

There are many linking moieties and methodologies for attaching fluorophore moieties to nucleotides. In certain aspects, the detectable moiety is a fluorescent organic dye derivatized for attachment to a γ-phosphate directly or via a linker. In general, nucleotide labeling can be accomplished using any of a large number of known nucleotide labeling techniques using known linkages, linking groups, and associated complementary functionalities. The linkage linking the fluorophore to the phosphate should be compatible with relevant polymerases.

In one embodiment, the linker is an alkylene group, such as a methylene or ethylene group. In this embodiment, the fluorophore linker is an alkylene group having between about 1 to about 50 carbon atoms, preferably about 10 to 30 carbon atoms and more preferably, about 15 to about 25 carbon atoms, optionally interrupted by heteroatom(s). In certain aspects, the linker has at least 1 positive or negative charge associated therewith.

C. Charged Moieties on the Base

In certain aspects, the base has a charged moiety appended thereto to increase or decrease molecular charge. In general, attaching one or more nucleotide charged moieties can be accomplished using any of a large number of known nucleotide labeling techniques using known linkages, linking groups, and associated complementary functionalities. Preferably, the linkage attaching the charged moiety and nucleotide should be compatible with relevant polymerases.

Preferably, the charged moieties are covalently linked to the 5-carbon of pyrimidine bases and to the 7-carbon of 7-deazapurine bases. Several suitable base labeling procedures have been reported that can be used with the present invention, e.g. Gibson et al., Nucleic Acids Research, 15: 6455-6467 (1987); Gebeyehu et al., Nucleic Acids Research, 15: 4513-4535 (1987); Haralambidis et al., Nucleic Acids Research, 15: 4856-4876 (1987); Nelson et al., Nucleosides and Nucleotides, 5(3) 233-241 (1986); Bergstrom, et al., JACS, 111, 374-375 (1989); U.S. Pat. Nos. 4,855,225, 5,231,191, and 5,449,767, each of which is incorporated herein by reference. Preferably, the linkages are acetylenic amido or alkenic amido linkages, the linkage between the charged moiety and the nucleotide base being formed by reacting an activated N-hydroxysuccinimide (NHS) ester of the charged moiety with an alkynylamino- or alkenylamino-derivatized base of a nucleotide.

D. Assay to Assess Charge

Those of skill in the art will readily recognize that various assays are easily implemented to assess the charge of the intact nucleotide phosphate and the cleaved pyrophosphate carrying a label. The following assay is just one of many available assays to calculate and assess the net charge on the γ-NP-Dye and the released PPi-F or P—F moiety.

In one instance, an assay is used to test for a change in the electric charge associated with a dye attached to the terminal phosphate of a nucleotide. For example, the charge switch is caused by cleavage of a phosphodiester bond that links the dye to the nucleotide. In one example, cleavage is catalyzed by snake venom phosphodiesterase. It will be appreciated by those of skill in the art that other enzymes, such as a DNA polymerase claimed herein, can also be used to demonstrate charge switching.

One assay for identifying an intact charge-switch nucleotide phosphate (NP) probe, includes a) contacting a sample comprising the intact charge-switch NP probe with an enzyme of the present invention to produce a phosphate detectable moiety; and b) applying an electric field to the sample, wherein the phosphate detectable moiety migrates to an electrode differently than the intact charge-switch NP probe.

IV. Mutant DNA Polymerases of this Invention

In one aspect, the invention provides purified DNA polymerases with charge-switch nucleotide interaction pockets that have been mutated to optimize polymerase activity for charge-switch nucleotides. Optionally, the charge-switch nucleotide interaction pocket is also mutated to decrease activity for non-charge-switch nucleotides. Optionally, the exonuclease domain is mutated to decrease exonuclease activity of the polymerase. Since most naturally occurring polymerases have limited activity for charge-switch nucleotides, such purified DNA polymerases considerably enhance the speed and accuracy of sequencing with charge-switch nucleotides.

A. DNA Polymerases Used as Parent Polymerases for Mutations

In preferred embodiments, the mutant DNA polymerase of this invention is derived from a φ29 DNA polymerase. Advantageously, φ29 polymerases exhibit strong strand displacement activity and exceptional processivity.

In addition to providing mutant φ29 DNA polymerases with increased polymerase activity for charge-switch nucleotides, the invention provides mutant forms of other polymerases from the φ29-type family. These phages are generally described by Salas, 1 The Bacteriophages 169, 1988. The structure of these DNA polymerases is extremely similar, with some differing by as few as 6 amino acid changes with 5 of those amino acids being replaced by similar amino acids. These polymerases have a highly active 3′-5′ exonuclease activity, but no 5′-3′ exonuclease activity. The φ29-type polymerases include those polymerases from Cp-1, PRD1, φ15, φ21, PZE, PZA, Nf, M2Y, B103, SFS, GA-1, Cp-5, Cp-7, PR4, PRS, PR722, and L17 phages.

In general, the teachings of the invention may be used to produce mutant DNA polymerases having increased polymerase activity for charge-switch nucleotides from any DNA polymerase that shares sufficient amino acid sequence homology to φ29 DNA polymerase to permit a person of ordinary skill in the art to identify one or more amino acid residue positions in the DNA polymerase that are analogous to amino acids within the charge-switch nucleotide interaction region of a φ29 DNA polymerase.

Parent DNA polymerases that may be modified to contain mutations in the charge-switch nucleotide interaction region include, but are not limited to, DNA polymerases from organisms such as Thermus flavus, Pyrococcus furiosus, Thermotoga neapolitana, Thermococcus litoralis, Sulfolobus solfataricus, Thermatoga maritima, E. coli phage T5, and E. coli phage T4. The DNA polymerases may be thermostable or not thermostable.

In certain embodiments, the parent polymerase can also be a T7 polymerase. T7 polymerase has a known 3D structure and is known to be processive. In order to operate in a strand-displacement mode, the polymerase requires a complex of three proteins: T7 polymerase+thioredoxin+primase (Chowdhury et al. PNAS 97:12469). In other embodiments, the parent polymerases can also be HIV RT and DNA Polymerase I.

Additionally, embodiments of the invention include some purified naturallγ-occurring DNA polymerases that have increased polymerase activity for charge-switch nucleotides. Such naturally-occurring DNA polymerases are structurally and functionally analogous to the mutant DNA polymerases explicitly described herein.

B. Mutations to Increase Charge-Switch Nucleotide Polymerase Activity

The mutant DNA polymerases of this invention contain mutations of amino acid residues in the charge-switch nucleotide interaction region. It is well known in the art that DNA polymerases undergo conformational changes upon binding of nucleotides during DNA synthesis and that structural alterations of the nucleotide can reduce binding. In fact, naturally occurring DNA polymerases preferentially incorporate unmodified nucleotides over corresponding modified nucleotides. The present invention is based on the discovery that mutations within the charge-switch nucleotide interaction region can increase activity for these modified nucleotides, presumably by restoring the “fit” between the binding pocket and the modified nucleotide.

As described in the above section, nucleotides can be modified in several ways to generate a “charge-switch nucleotide”. In especially preferred embodiments, the nucleotides are coupled to a detectable moiety at the γ-phosphate and DNA polymerases of the invention have mutations in regions of the nucleotide binding pocket which closely interact with the phosphate detectable moiety of the nucleotide. In other preferred embodiments, the modified nucleotides have both a terminal phosphate with a detectable moiety and other modifications as described in the preceding section. In these cases, the DNA polymerase is preferentially mutated in regions of the nucleotide binding pocket which interact with any of the modified aspects of the nucleotide. For example, the modified nucleotide may have a label attached to the sugar and thus, the mutant DNA polymerase will have mutations in the sugar interaction region. In another instance the modified nucleotide may have both a label attached to the γ-phosphate and a charged moiety attached to the base and thus the mutant DNA polymerase will have mutations in both the nucleotide γ-phosphate interaction region and the base region.

Mutant DNA polymerases of the invention have one or more mutations at amino acid residue positions within the charge-switch nucleotide interaction region of a given DNA polymerase. In some embodiments, there are at least two mutations. In other embodiments, there are at least three mutations. These mutations may be in the γ-phosphate region, the sugar region, the base region, or in combinations thereof. Such mutations are usually, although not necessarily, substitution mutations. Several different amino acid residues may be substituted at a given position of a parent enzymes so as to give rise to mutations that enhance charge-switch nucleotide polymerase activity. The amino acid residues at a given residue position within the charge-switch nucleotide interaction region may be systematically varied so as to determine which amino acid substitutions are effective. Preferably, the mutations are non-conservative mutations.

Specific Mutations

In certain embodiments, the DNA polymerase has mutations in the nucleotide γ-phosphate region. Especially preferred site(s) for mutation of φ29 polymerase are Ile-115, His-116, Ile-179, Gln-180, Phe-181, Lys-182, Gln-183, Gly-184, Leu-185, Val-247, Phe-248, Asp-249, Val-250, Asn-251, Ser-252, Leu-253, Pro-255, Ala-256, Gly-350, Leu-351, Lys-352, Phe-353, Lys-354, Ala-355, Thr-356, Thr-357, Gly-358, Leu-359, Phe-360, Lys-361, Asp-362, Phe-363, Ile-364, Asp-365, Lys-366, Trp-367, Thr-368, Tyr-369, Ile-370, Lys-371, Thr-372, Thr-373, Ser-374, Glu-375, Gly-376, Ala-377, Ile-378, Lys-379, Gln-380, Leu-381, Ala-382, Lys-383, Leu-384, Met-385, Leu-386, Asn-387, Asp-458, Ser-459, Trp-483, Ala-484, His-485, Glu-486, Ser-487, Thr-488, Phe-489, Ile-501, Gln-502, Asp-503, Ile-504, Tyr-505, Met-506, Lys-507, Glu-508, Val-509, Asp-510, and combinations thereof. In preferred embodiments, Lys-383 is mutated; preferably, to Ala-383.

In other embodiments, the DNA polymerase has mutations in the sugar (ribose) interaction region. Especially preferred site(s) for mutation of φ29 DNA polymerases are Tyr254, Tyr390, Thr457, and combinations thereof.

In another embodiment, each of the foregoing interaction regions are mutated in combination.

In still other embodiments, the DNA polymerase has mutations in the nucleobase interaction region. Especially preferred site(s) for mutation of φ29-type DNA polymerases are Thr-117, Val-118, Ile-119, Tyr-120, Asp-121, Asp-200, Ile-201, Ile-202, Thr-203, Thr-204, Lys-205, Lys-206, Phe-207, Lys-208, Lys-209, Ala-225, Tyr-226, Arg-227, Gly-228, Gly-229, Phe-230, Thr-231, Trp-232, Leu-233, Asn-234, Asp-235, Arg-236, Ser-388, Leu-389, Tyr-390, Gly-391, Gln-497, Lys-498, Thr-499, Lys-512, Leu-513, Val-514, Glu-515, Gly-516, Ser-517, and combinations thereof.

Additional Factors Influencing the Identity of Mutations

It will be appreciated by persons skilled in the art of molecular biology that the charge-switch nucleotide interaction region of a given DNA polymerase is defined with respect to a specific modified nucleotide. Changes in one or more of the following parameters of the structure of a modified nucleotide may alter the identity of the amino acid residues that form the charge-switch nucleotide interaction site of a given DNA polymerase: (1) identity of the base, (2) the site of attachment of the charge on the nucleotide base, (3) the identity of the linker joining the phosphate to the florescent dye, (4) identity of the charged group on the base, and the (5) the identity of the fluorescent dye.

It will further be appreciated by those of skill in the art that the mutations within the charge-switch nucleotide interaction binding pocket which confer the greatest amounts of increased activity will vary depending on the particular modifications to the nucleotides, the type of label linked to the terminal phosphate, the type of linker, modifications to the nucleobase, etc.

C. Methods for Making Mutations

The residues lining the charge-switch nucleotide interaction region will vary depending on the particular DNA polymerase and in some degree, will vary depending in the particular modified nucleotide. The residue can be any residue identified as one that is in close proximity to or interacts with charge-switch nucleotides. Such residues can be identified by any method known to those of skill in the art for predicting and modeling secondary and tertiary protein structure.

In instances where it is difficult to obtain structural information and where large regions of homology can be found between these different DNA polymerases, the determination of analogous amino acid residues between different DNA polymerases can be used to identify residues lining the charge-switch nucleotide interaction region. A large compilation of the amino acid sequences of DNA polymerases from a wide range of organism and homology alignments between the sequences can be found in Braithwaite and Ito, Nucl. Acids Res. 21(4):787-802 (1993) and is useful for such purposes.

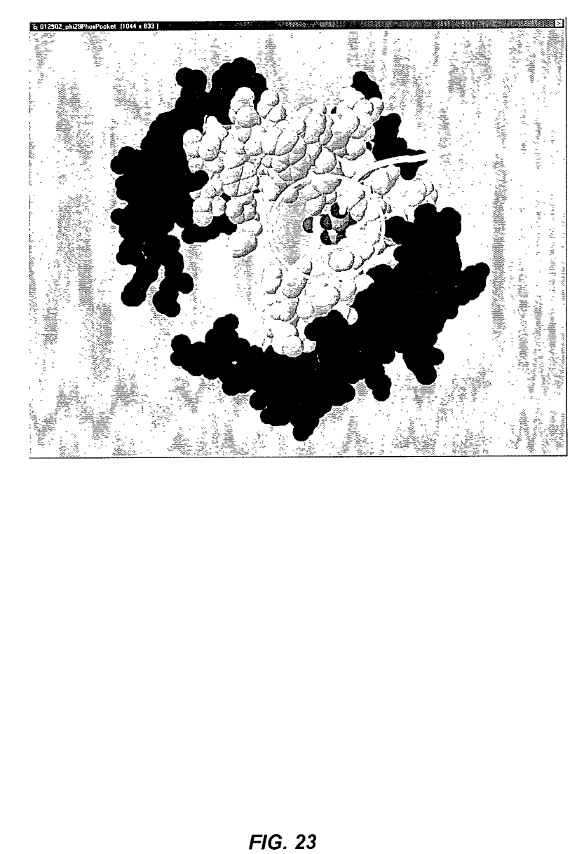

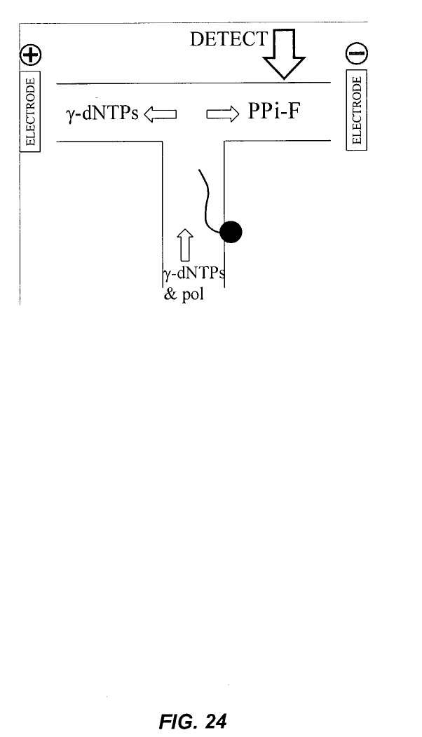

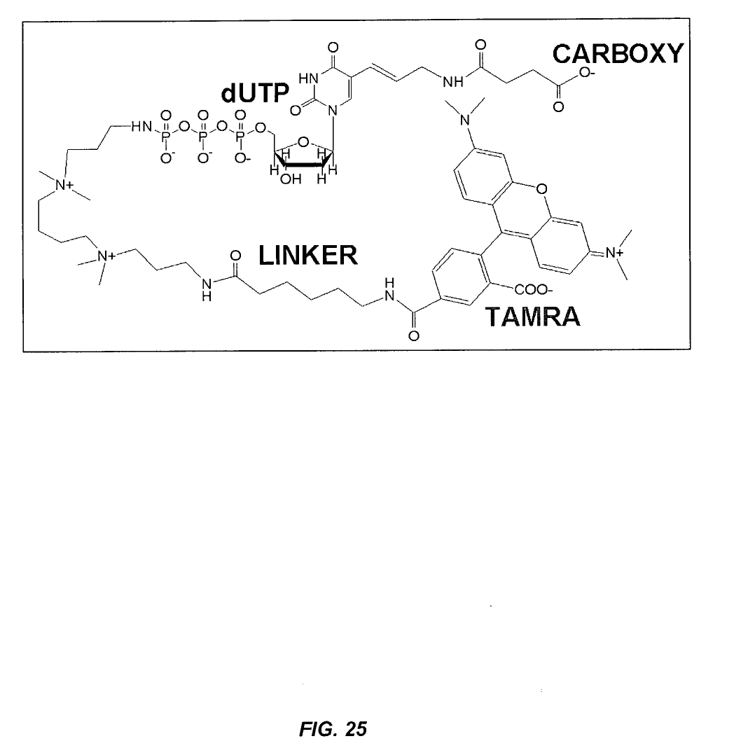

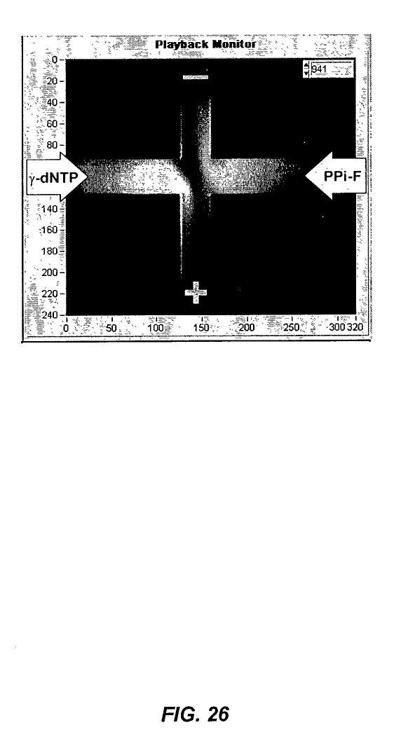

A computer model of the φ29 polymerase has been developed (FIG. 23). By predicting the location of the γ-phosphate nucleotide binding pocket, the base interaction region, and the sugar interaction region, the model provides guidance in making mutations in DNA polymerase that influence activity for charge-switch nucleotides. The model successfully explains the behavior of many site-directed mutations reported in the literature. Based on the model, sequences of exemplary mutant φ29 DNA polymerases have been identified and are set forth in Table 1 (SEQ ID NOs:4-36). Columns 1 and 2 of Table 1 set forth below specify the WT residues that are part of the nucleotide γ-phosphate interaction region. Each column to the right describes a particular mutated sequence by specifying the number of residues that are mutated relative to WT and indicating which of the nucleotide γ-phosphate interaction region residues have been mutated.

SEQ ID NO:

| | | | | | | | | | | | | | | | | |
|---|---|---|---|---|---|---|---|---|---|---|---|---|---|---|---|---|
| 4 | 5 | 6 | 7 | 8 | 9 | 10 | 11 | 12 | 13 | 14 | 15 | 16 | 17 | 18 | 19 | 20 |

NUMBER OF MUTATIONS RELATIVE TO WILD-TYPE SEQUENCE

| position | WT residue | 2 | 2 | 1 | 1 | 2 | 1 | 1 | 2 | 2 | 2 | 3 | 2 | 1 | 1 | 1 | 2 | 2 |
|---|---|---|---|---|---|---|---|---|---|---|---|---|---|---|---|---|---|---|
| 115 | ILE | | | | | | | | | | | | | | | | | |
| 116 | HIS | | | | | Ile | | | | | | | | | | | | |
| 179 | ILE | | | | | | | | | | | | | | | | | |
| 180 | GLN | | | | | | | | | | | | | | | | | |
| 181 | PHE | | | | | | | | | | | | | | | | | |
| 182 | LYS | | | | | | | | | | | | | | | | | |
| 183 | GLN | | Trp | | | | | | | | | | | | | | | |
| 184 | GLY | | | | | | | | | | | | | | | | | |
| 185 | LEU | | | | | | | | | | Lys | | | | | | | |
| 247 | VAL | | | | | | | | | | | Gly | | | | | Trp | Asn |
| 248 | PHE | | | | | | | | | | | | | | | | | |
| 249 | ASP | | | | | | | | | | Val | | | | | | | |
| 250 | VAL | | | | | | | | Met | | | | | | | | | |
| 251 | ASN | | | | | | | Ala | | | | | | | | | | |
| 252 | SER | | | | | | | | | | | | | | | | | |
| 253 | LEU | | | | | | | | | | | | | | | | | |
| 255 | PRO | | Val | Ser | | | | | | | | | | | | | | |
| 256 | ALA | | | | | | | | | | | | | | | | | |
| 350 | GLY | | | | | | | | | | | | | | | | | |
| 351 | LEU | | | | | | | | | | | | | | | | | |
| 352 | LYS | | | | | | | | | | | | | | | | | |
| 353 | PHE | | | | | | | | | | | | | | | | | |
| 354 | LYS | | | | | | | | | | | | | | | | | |
| 355 | ALA | | | | | | | | | | | | | | | | | |
| 356 | THR | | | | | | | | | | | | | | | | | |
| 357 | THR | | | | | | | | | | | | | | | | | |
| 358 | GLY | Pro | | | | | | | | | | | | | | | | |
| 359 | LEU | | | | | | | | | Phe | | | | | | | | |
| 360 | PHE | | | | | | | | | | | | | | | | | |
| 361 | LYS | | | | | | | | | | | | | | | | | |
| 362 | ASP | | | | | | | | | | | | | | | | | |
| 363 | PHE | | | | | | | | | | | | | | | | | |
| 364 | ILE | | | | | | | | | Ser | | Leu | Tyr | | | | | |
| 365 | ASP | | | | | | | | | | | | | | | | | |
| 366 | LYS | | | | | | | | | | | | | | | | | |
| 367 | TRP | | | | | | | | | | | | | | | | | |
| 368 | THR | | | | | | | | | | | | Trp | | | | | |
| 369 | TYR | Val | | | | | | | | | | | | | | | | |
| 370 | ILE | | | | | | | | | | | | | | | | | |
| 371 | LYS | | | | | | | | | | | | | | | | | |
| 372 | THR | | | | | | | | | | | | | | | | | |
| 373 | THR | | | | | | | | | | | | | | | | | |
| 374 | SER | | | | | | | | | | | | | | | | | |
| 375 | GLU | | | | | | | | | | | | | | | | | |
| 376 | GLY | | | | | | | | | | | | | | | | | |
| 377 | ALA | | | | | | | | | | | | | | | | | |
| 378 | ILE | | | | | | | | | | | | | | | | | |
| 379 | LYS | | | | | | | | | | | | | | | | | |
| 380 | GLN | | | | | | | | | | | | | | | | | |
| 381 | LEU | | | | | | | | | | | | | | | | | |
| 382 | ALA | | | | | | | | | | | | | | | | | |
| 383 | LYS | | | | | | | | | | | | | | | | | |
| 384 | LEU | | | | | | | | | | | | | | | Thr | | |
| 385 | MET | | | | | | | | | | | | | | | | | |
| 386 | LEU | | | | | | | | | | | Cys | | | | | | |
| 387 | ASN | | | | | | | | | | | | | | | | | |
| 458 | ASP | | | | | | | | | | | | | | | | | |
| 459 | SER | | | | | Thr | | | | | | | | | | | | |
| 483 | TRP | | | | | | | | | | | | | | | | | |
| 484 | ALA | | | | | | | | | | | | | | | | | |
| 485 | HIS | | | | | | | | | | | | | | | | His | |
| 486 | GLU | | | | | | | | | | | | | | | | | |
| 487 | SER | | | | | | | | | | | | | | | | | |
| 488 | THR | | | | | | | | Leu | | | | | | | | | |
| 489 | PHE | | | | | | Asn | | | | | | | | | | | |
| 501 | ILE | | | | | | | | | | | | | | | | | |
| 502 | GLN | | | | | | | | | | | | | | Met | | | |
| 503 | ASP | | | | | | | | | | | | | | | | | Asn |
| 504 | ILE | | | | | | | | | | | | | | | | | |
| 505 | TYR | | | | | | | | | | | | | | | | | |
| 506 | MET | | | | | | | | | | | | | | | | | |
| 507 | LYS | | | | Ser | | | | | | | | | | | | | |
| 508 | GLU | | | | | | | | | | | | | Pro | | | | |
| 509 | VAL | | | | | | | | | | | | | | | | | |
| 510 | ASP | | | | | | | | | | | | | | | | | |

| | | SEQ ID NO: | | | | | | | | | | | | | | | |
|---|---|---|---|---|---|---|---|---|---|---|---|---|---|---|---|---|---|
| | | 21 | 22 | 23 | 24 | 25 | 26 | 27 | 28 | 29 | 30 | 31 | 32 | 33 | 34 | 35 | 36 |
| | | NUMBER OF MUTATIONS RELATIVE TO WILD-TYPE SEQUENCE | | | | | | | | | | | | | | | |
| position | WT residue | 2 | 1 | 1 | 2 | 2 | 1 | 1 | 1 | 4 | 2 | 1 | 4 | 1 | 4 | 4 | 2 |

| | | | | | | | | | | | | | | | | | |
|---|---|---|---|---|---|---|---|---|---|---|---|---|---|---|---|---|---|
| 115 | ILE | | | | | | | | | Met | | | | | | | |
| 116 | HIS | | | | Ser | | | | | | | | | | | | |
| 179 | ILE | | | | | | | | | Met | | | | | | | |
| 180 | GLN | | | | | | | | | | | | | | Thr | | |
| 181 | PHE | | | | | | Val | | | | | | | | | | |
| 182 | LYS | | | | | | | | | | | | | | | | |
| 183 | GLN | | | | | | | | | | | | | | | | |
| 184 | GLY | | | | | | | | | | | | | | | | |
| 185 | LEU | | | | | | | | | | | | | | | | |
| 247 | VAL | Pro | Glu | | | | | | | | | | | | | | |
| 248 | PHE | | | | | | | Asp | | | | | | | | | |
| 249 | ASP | | | | | | | | | | | | | Ile | | | |
| 250 | VAL | | | | | | | | | | | | | | | | |
| 251 | ASN | | | | | | | | | | | | | | | | |
| 252 | SER | | | | | | | | | | | | | | | Gly | |
| 253 | LEU | | | | | | | | | | | | Asn | | | | |
| 255 | PRO | | | | | | | | | | | | | | | | |
| 256 | ALA | | | | | | | | | | | | | | | | |
| 350 | GLY | | | | | | | | | | | | | | | | |
| 351 | LEU | | | | | | | | | | | | | | | | |
| 352 | LYS | | | | | | | | | | | | | | | | |
| 353 | PHE | | | | | | | | | | | | | | | | |
| 354 | LYS | | | | | | | | | | | | | | | | |
| 355 | ALA | | | | | | | | | | | | | | | | |
| 356 | THR | | | | | | | | | | | | | | | | |
| 357 | THR | | | | | | | | | | | | | | | | |
| 358 | GLY | | | | | | | | | | | | | | | | |
| 359 | LEU | | | | | | | | | | | | | | | | |
| 360 | PHE | | | | | | | | Arg | | | | | | | | |
| 361 | LYS | | Asp | | | | | | | | | | | | | Asn | |
| 362 | ASP | | | | | | | | | | | | Met | | | | |
| 363 | PHE | Pro | | | | | | | | | | | | | | | |
| 364 | ILE | | | | | | | | | | | | | | | | |
| 365 | ASP | | | | | | | | | | | | | | | | |
| 366 | LYS | | | | | | | | | | | | | | | Trp | |
| 367 | TRP | | | | | | | | | | | | | | | | |
| 368 | THR | | | | | | | | | | | | | | | | |
| 369 | TYR | | | | | | | | | | | | | | Cys | | |
| 370 | ILE | | | | | | | | | | | | | | | | |
| 371 | LYS | | | | | | | | | | | | | | | | |
| 372 | THR | | | | | | | | | | | | | | | | |
| 373 | THR | | | | | | | | | | | | | | | | |
| 374 | SER | | | | | | | | | | | | | | | | |
| 375 | GLU | | | | | | | | | | | | | | | | |
| 376 | GLY | | | | | | | | | Ser | | | | | | | |
| 377 | ALA | | | | | Asn | | | | | | | | | | | |
| 378 | ILE | | | | | | | | | | | | | | | | |
| 379 | LYS | | | | | | | | | | | | | | | | |
| 380 | GLN | | | | | | | | | | | | | | | | |
| 381 | LEU | | | Phe | | | | | | | | | | | | | Ile |
| 382 | ALA | | | | | | | | | | | | | | | | |
| 383 | LYS | | | | | | | | | | | | | | | | |
| 384 | LEU | | | | | | | | | | | | | | | | Gln |
| 385 | MET | | | | | | | | | | | | | | Tyr | | |
| 386 | LEU | | | | | | | | | | | | | | | | |
| 387 | ASN | | | | | | | | | | Cys | | | | | | |
| 458 | ASP | | | | | | | | | | | | | | | | |
| 459 | SER | | | | | | | | | | | | | | | | |
| 483 | TRP | | | | Val | | | | | | | | | | | | |
| 484 | ALA | | | | | | | | | | | | | | | Met | |
| 485 | HIS | | | | | | | | | | | | | | Met | | |
| 486 | GLU | | | | | | | | | | | | | | | | |
| 487 | SER | | | | | | | | | | | | | | | | |
| 488 | THR | | | | | | | | | | | | | | | | |
| 489 | PHE | | | | | | | | | | | | | | | | |
| 501 | ILE | | | | | | | | | | | | Ile | | | | |
| 502 | GLN | | | | | | | | Phe | | | | | | | | |
| 503 | ASP | | | | | | | | | | | | | | | | |
| 504 | ILE | | | | | | | | | | | | | | | | |
| 505 | TYR | | | | | | | | | | | | | | | | |
| 506 | MET | | | | | | | | | | | Phe | | | | | |
| 507 | LYS | | | | | Leu | | | | Met | | | Asp | | | | |
| 508 | GLU | | | | | | | | | | | | | | | | |
| 509 | VAL | | | | | | | | | | | | | | | | |
| 510 | ASP | | | | | | | | | | | | | | | | |

Although the computer model of the φ29 polymerase is believed to be an accurate three-dimensional structural model, it should in no way be considered as limiting the present invention. Those of skill in the art will understand that the various embodiments of the invention may be practiced regardless of the model used to described the theoretical aspects of the invention.

The mutations described above can be generated using any method typically used by those of skill in the art to introduce mutations at specific residues. Such methods are well described in Sambrook et al., Molecular Cloning: A Laboratory Manual, Cold Spring Harbor Laboratory Publications, Cold Spring Harbor, N.Y. (1982).

DNA Polymerases

Numerous genes encoding DNA polymerases have been isolated and sequenced. This sequence information is available on publicly accessible DNA sequence databases such as GENBANK. A large compilation of the amino acid sequences of DNA polymerases from a wide range of organism can be found in Braithwaite and Ito, Nucl. Acids Res. 21(4):787-802 (1993). This information may be used in designing various embodiments of DNA polymerases of the invention and polynucleotides encoding these enzymes. The publicly available sequence information may also be used to clone genes encoding DNA polymerases through techniques such as genetic library screening with hybridization probes.

Genes encoding parent DNA polymerase may be isolated using conventional cloning techniques in conjunction with publicly-available sequence information. Alternatively, many cloned polynucleotide sequences encoding DNA polymerases have been deposited with publicly-accessible collection sites, e.g., the American type culture collection deposit accession number ATCC 40336 is a phage clone of Taq DNA polymerase.

D. Additional Mutations

The mutant DNA polymerases of the invention can comprise numerous mutations in addition to those for increasing charge-switch nucleotide polymerase activity. These secondary mutations may be either inside or outside the charge-switch nucleotide interaction region. Secondary mutations can be selected so as to confer some useful property on the mutant DNA polymerase. For example, additional mutations may be introduced to increase thermostability, decrease thermostability, increase processivity, decrease processivity, decrease 3′-5′ exonuclease activity, increase 3′-5′ exonuclease activity, decrease 5′-3′ exonuclease activity, increase 5′-3′ exonuclease activity, increase incorporation of dideoxynucleotides, and decrease activity towards non-charge-switch nucleotides.

In preferred embodiments, the subject mutant DNA polymerases comprise one or more secondary mutations that reduce or eliminate 3′-5′ exonuclease activity, such as mutations in Asn-62 and Thr-15. Most preferably, the mutations to eliminate exonuclease activity are N62D or T15I. DNA polymerases that are deficient in 3′-5′ exonuclease activity are particularly suitable for PCR and for chain termination polynucleotide sequencing. Mutations that reduce 3′-5′ exonuclease activity in DNA polymerase are well known to persons of ordinary skill in the art. Detailed guidance on how to introduce mutations that reduce 3′-5′ exonuclease activity can be found, among other places, in U.S. Pat. No. 4,795,699 (Tabor); U.S. Pat. Nos. 5,541,099; 5,489,523; and Bernad et al., Cell 59:219-288 (1989).

Preferably, for single molecule sequencing applications as described in U.S. Pat. No. 6,255,083, as well as the other applications incorporated by reference, the subject DNA polymerases comprise one or more secondary mutations that reduce 3′-5′ exonuclease activity yet retain strand displacement activity. For example, the mutation (N62D) eliminates exonuclease while preserving strand-displacement synthesis (de Vega et al. EMBO J 15:1182). Exonuclease activity allows newly-added bases to be removed from the primer strand and then added back by polymerase. Thus, the same base can be added twice in succession, a characteristic which is not desirable for charge-switch sequencing.

In other embodiments, the subject DNA polymerases comprise mutations that decrease non-charge-switch polymerase activity. Mutations with this effect are well known in the art.

In especially preferred embodiments, the subject DNA polymerases comprise mutations in the charge-switch nucleotide interaction region, mutations that decrease exonuclease activity, and mutations that decrease non-charge-switch nucleotide polymerase activity.

V. Methods of Generating Mutant DNA Polymerases of the Invention

A. Overview

In one aspect, the present invention relates to methods for the production of nucleic acid fragments encoding mutant proteins having charge-switch nucleotide polymerase activity. Typically, such methods comprise providing a polynucleotide, mutating the polynucleotide to generate a library of mutated polynucleotides, and selecting a polynucleotide encoding a polypeptide with improved charge-switch nucleotide polymerase activity. In some embodiments, the methods also comprise selecting mutated polypeptides with decreased activity for non-charge-switch nucleotides and decreased exonuclease activity.

B. Parent Polynucleotides

The polynucleotide used as starting material can encode any polymerase known to those of skill in the art with properties which make it suitable for the desired uses of charge-switch nucleotides. In preferred embodiments, the initial polynucleotide encodes a DNA polymerase from the φ29-type family. φ29-type polymerases include those polymerases from Cp-1, PRD1, φ15, φ21, PZE, PZA, Nf, M2Y, B103, SFS, GA-1, Cp-5, Cp-7, PR4, PRS, PR722, and L17 phages. Most preferably, the polymerase is a φ29 polymerase, which has strong strand displacement activity and is highly processive. In other preferred embodiments, the polynucleotides encode HIV RT, T7 polymerase, or DNA Polymerase I.

Native polynucleotide sequence encoding active polymerase can be used as the starting material for methods of this invention. However, in preferred embodiments, the parent polynucleotide encodes an inactive polymerase. Elimination of background activity from weakly-active enzymes allows desired mutants to be unambiguously detected during the screen. In particularly preferred embodiments, the parent polynucleotide encodes an inactive polymerase and lacks exonuclease activity. In other embodiments, the parent polynucleotide encodes an active polymerase.

C. Methods of Generating a Library of Mutants

Methods of generating a library of mutants are well known to those of skill in the art. In preferred embodiments, the polynucleotide is mutated via in vitro or in vivo recombination, site-directed mutagenesis, error-prone PCR, site-saturation mutagenesis, or gene shuffling recombination.

In one embodiment, the original polynucleotide is systematically mutated at specific amino acids in the charge-switch nucleotide interaction region.

In other preferred embodiments, the polynucleotides are first mutated using a method which randomly introduces mutations, such as error-prone PCR; screened for desired activity; mutated using a method which introduces all possible mutations at the mutant amino acids which confer the desired activity, such as site-saturation mutagenesis; and then recombined or further mutated by methods such as the StEP (staggered extension process) method or other single-site or multi-site mutagenesis methods. Site-directed mutagenesis techniques are well known in the art as exemplified by U.S. Pat. Nos. 4,711,848; 4,873,192; 5,071,743; 5,284,760; 5,354,670; 5,556,747; Zoller and Smith, Nucleic Acids Res. 10:6487-6500 (1982), and Edelman et al. DNA 2:183 (1983). Detailed protocols for site-directed mutagenesis are also given many general molecular biology textbooks such as Sambrook et al. Molecular Cloning a Laboratory Manual 2nd Ed. Cold Spring Harbor Press, Cold Spring Harbor (1989), Ausubel et al. Current Protocols in Molecular Biology, (current edition). Additionally, many textbooks on PCR (the polymerase chain reaction), such as Diefenbach and Dveksler, PCR Primer: A Laboratory Manual, Cold Spring Harbor Press, Cold Spring Harbor, N.Y. (1995), describe methods of using PCR to introduce mutations.

In other preferred embodiments, shuffling methods such as those described in U.S. Pat. No. 6,117,679, issued to Stemmer et al. are used to generate additional mutants from mutant polynucleotides with increased charge-switch nucleotide polymerase activity and/or polymerases with natural activity for charge-switch nucleotides. In some cases, two polynucleotides encoding mutant versions of the same polymerase are shuffled. In other cases, a polynucleotide encoding one type of polymerase and a polynucleotide encoding a different polymerase with sufficient nucleotide homology to permit shuffling and are shuffled. Gene shuffling utilizes naturally occurring nucleotide substitutions among family genes as the driving force for in vitro evolution. (see, Chang, C.-C., Chen, T. T., Cox, B. W., Dawes, G. N., Stemmer, W. P. C., Punnonen, J., and Patten, P. A. Evolution of a cytokine using DNA family shuffling. Nat. Biotechnol., 17, 793-797. (1999); Hansson, L. O., B-Grob, R., Massoud, T., and Mannervik, B. Evolution of differential substrate specificities in Mu class glutathione transferases probed by DNA shuffling. J. Mol. Biol., 287, 265-276. (1999); and Kikuchi, M., Ohnishi, K., and Harayama, S. An effective family shuffling method using single-stranded DNA. Gene, 243, 133-137. (2000)).

In certain embodiments, the present invention also relates to a method of repeated cycles of mutagenesis, nucleic acid mutation and selection which allow for the creation of mutant proteins having enhanced charge-switch nucleotide polymerase activity.

D. Selection of Mutants with Desired Activity

Polynucleotides with desired activity can easily be selected using standard methods. Activity for non-charge-switch nucleotides can be detected using standard assays for incorporation of dNTPs. Activity for charge-switch nucleotides can be detected using standard methods for detection of the detectable moieties of the charge-switch nucleotides, PCR-based assays for amplification of newly synthesized strands of DNA containing charge-switch nucleotides, or any other methods known to those of skill in the art. Since the activity of polymerases can differ depending on the precise properties of the particular charge-switch nucleotide, it is desirable to test a variety of different types of charge-switch nucleotides as substrates. Exonuclease activity can measured using assays well known in the art.

VI. Methods of DNA Sequencing Using Mutant DNA Polymerases of this Invention

In other aspects, the invention comprises methods of using the optimized charge-switch nucleotides of this invention in any assay, test, or method that requires the synthesis of sequences containing charge-switch nucleotides or where it would be useful to have sequences containing charge-switch nucleotides. Due to their unique charge-switch properties, the polymerases of this invention have utility in any molecular biology applications where it would either be advantageous or necessary to separate unincorporated dNTPs from cleaved pyrophosphate. In particular, these polymerases would be useful in methods where rapid, highly processive DNA synthesis is desired.

More generally, the mutant polymerases of this invention can be substituted for the corresponding parent DNA polymerase in most procedures that employ DNA polymerases, particularly those where activity for charge-switch nucleotides is desired.

In preferred embodiments, the polymerases of this invention are used in methods for single molecule real-time DNA sequencing. In one embodiment, the method comprises: a) immobilizing a complex comprising a purified φ29-type DNA polymerase or a target nucleic acid onto a solid phase in a single molecule configuration, wherein the purified φ29-type DNA polymerase has at least one amino acid change as defined with respect to a naturally occurring φ29-type DNA polymerase, wherein the at least one amino acid change is in the charge-switch interaction region, the purified φ29-type DNA polymerase having increased activity for a charge-switch nucleotide;

b) contacting the complex with a primer nucleic acid which complements a region of the target nucleic acid of the region to be sequenced and a sample stream comprising a target nucleic acid when the purified DNA polymerase is immobilized or the purified DNA polymerase when the target nucleic acid is immobilized and a charge-switch nucleotide having a detectable moiety, wherein the detectable moiety is released as a charged detectable moiety when the charge-switch nucleotide is incorporated into the primer nucleic acid wherein the solid phase is attached to a flowcell having an inlet port and an outlet port;

c) applying an energy field to the sample stream; and

d) detecting the charged detectable moiety, thereby sequencing the target nucleic acid.

In other preferred embodiments, the polymerases of this invention are used in methods described in issued U.S. Pat. No. 6,255,083, which is hereby incorporated by reference. Briefly, in one embodiment, the invention comprises a method of genotyping or sequencing a target nucleic acid comprising the steps of;

a) immobilizing onto a solid support a complex comprising a target nucleic acid, a primer nucleic acid which complements a region of the target nucleic acid, and at least one mutant DNA polymerase of this invention;

b) contacting the immobilized complex with at least one type of labeled nucleotide triphosphate (NTP), wherein each type of NTP is differently labeled with a detectable label which is released when the NTP is incorporated, and

c) detecting the incorporation of a labeled NTP into a single molecule of the primer by detecting a unique label released from the labeled NTP, to genotype or to sequence the target nucleic acid.

VII. Kits

As described above, mutant DNA polymerases with increased charge-switch nucleotide activity have numerous molecular biology applications. Thus, the invention also provides kits comprising DNA polymerases and charge-switch nucleotides. Such kits can be prepared from polymerases described herein together with readily available materials and reagents. Kits preferably contain detailed instructions for how to perform the procedures for which the kits are adapted. A wide variety of kits can be prepared, depending on the intended user of the kit and the particular need of the user.

### EXAMPLES

### Example 1

### Methods of Screening for Polypeptides with Charge-Switch Nucleotide Polymerase Activity

This example describes methods for generating and identifying mutant DNA polymerases with activity for charge-switch nucleotides and the approach for developing such methods.

1. Introduction

DNA polymerases that efficiently incorporate “charge-switched” γ-phosphate-labeled dNTPs for single-molecule DNA sequencing have been developed. A variety of dNTPs are synthesized to provide different charge-switch configurations. Polymerase variants are selected for utilization of the charge-switch nucleotides using the described directed evolution methods.

Nucleotide Chemistry

The effect of different nucleotide chemistry is investigated by constructing dNTPs with various structures. For example, four dNTPs (ACGT) are labeled on the γ-phosphate with dyes of differing structure and charge for use in the polymerase selections. The nucleobase moieties are either unlabeled or tagged with electrically charged groups in different charge-switching configurations. Some configurations maximize the charge difference between γ-dNTP and PP—F, which is good for electrosorting microfluidics. Both aliphatic and peptide linkers are used to connect the dyes to the γ-P. The linkers have different numbers of charged groups to compensate the different dye charges as required for charge switching. Directional coupling of peptide linkers to the nucleotide is accomplished using a peptidase to “deprotect” the N-terminus of the linker after it is coupled to the γ-P.

Polymerase Libraries Mutation, and Recombination

An iterative approach to directed evolution is used to construct polymerase libraries containing mutant enzymes. Mutations are constructed in a DNA polymerase, such as T7 polymerase, by error-prone PCR using a kit from Stratagene designed especially for directed evolution applications. After screening for, and characterizing, improved enzymes, mutant amino acid positions are saturation-mutated to all possible substitutions using degenerate oligonucleotides in a published modification of Stratagene's QuikChange method. Selected mutants are recombined and/or further mutated by the StEP (staggered extension process) method or by the same QuikChange modified method as used for saturation mutagenesis.

High-Throughput Screening and Clone Selection

A PCR-based assay is used to identify polymerases with activity towards charge-switch nucleotides. This assay has sufficient power to detect one active polymerase in a pool of up to 1E06 inactive enzymes, an ability which enables single-tube screening of entire libraries comprising ˜1E06 unique clones. Quantitative TaqMan PCR is used to estimate the number of active clones in a given library under various assay conditions (γ-dNTP concentrations, reaction times). The libraries are screened in high-throughput mode to isolate individual clones using a pool deconvolution scheme. Automated pipetting robots are used to improve laboratory productivity and assay reliability for protein purification and assay setup. Isolated clones are sequenced and functionally characterized. Polymerases are adapted separately to γ-labels and charged nucleobase groups, then the different mutations are recombined to select for tolerance to both moieties as necessary. In one embodiment, polymerase incorporation rate of 10 nt/sec at 1-10 μM of each nucleotide is used as a standard to select clones. Polymerases are adapted to the various charge-switched γ-dNTPs. Nucleotides that maximize the charge-switch magnitude are preferred.

2. Significance

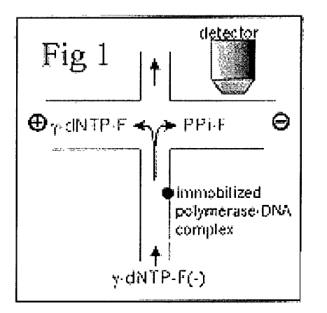

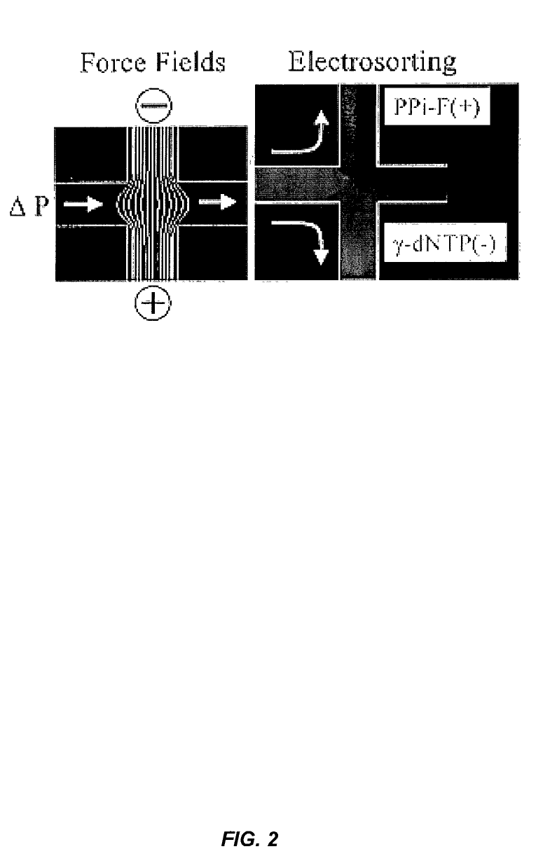

Electrosorting. As described above, the γ-label is cleaved from γ-dNTPs by a DNA polymerase of the present invention. There is a change in electric charge between an intact γ-dNTP—F and its cleavage product PPi-F, and this change is sensitive to the ionic composition of the medium and to charged groups on the γ-label and/or nucleobase. One approach to single-molecule sequencing utilizes charge switching to separate PPi-F groups from excess γ-dNTPs in a microfluidics sorting system. In a preferred embodiment, the γ-dNTP is negative and the PPi-F positive. This embodiment is illustrated in FIG. 1. A polymerase-DNA complex is immobilized just upstream from a channel intersection. An electric field at the intersection drives intact γ-dNTPs into a first microchannel toward the anode, while PPi-F molecules are driven toward the cathode into a second channel where they are detected. Each of the γ-dNTPs is labeled with a different dye, enabling real-time sequencing as successive PPi-γ-Dye molecules flow through the detection channel. By electrically sorting oppositely-charged molecules in this manner, the cleaved PPi-γDye molecules are detected in isolation without interference from unincorporated γ-dNTPs and without illuminating the polymerase-DNA complex. This embodiment is facilitated by a microfluidics model showing that oppositely-charged species (+1/−1) can be efficiently separated in microchannels (FIG. 2).

Single molecule immobilization. One approach is to immobilize exactly one histidine-tagged polymerase molecule on an individual nanofabricated nickel post smaller than the polymerase itself (<10 nm), so that only one enzyme will bind. The immobilized polymerase will select a DNA template from solution and begin to sequence it. In one aspect, 1 polymerase-DNA complex can be present per microchannel for successful sequencing.

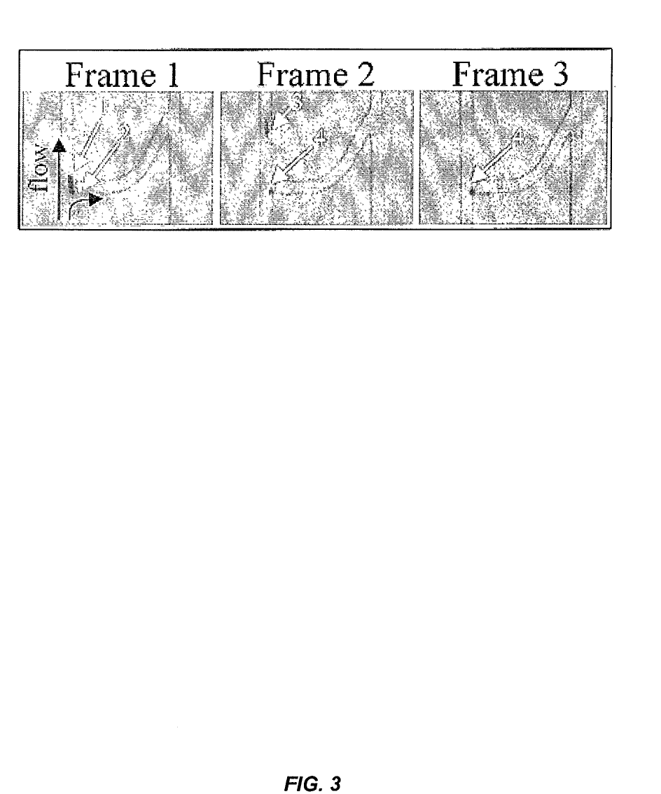

Another approach is to immobilize single DNA molecules on magnetic microbeads which are trapped on the channel wall (FIG. 3). The DNA (20-40 kb) is thereby positioned for sequencing in a flowstream containing DNA polymerase and γ-dNTPs. When done, the bead is flushed out and a new bead is trapped for the next round of sequencing. Floweell lifetime is not limited by enzyme survival and enzyme processivity is less important for achieving long reads when the DNA is immobilized.

2.1 Single Molecule Detection

Dye photodearadation and blinking. Single molecule fluorescence detection has been practiced now for over ten years (http://www.wiley-vch.de/berlin/journals/singmol/Single Molecules). It is straightforward to detect individual dye molecules. However, for DNA sequencing, it is highly preferable to have efficient detection of all signal molecules regardless of the particular sequencing scheme used. It is therefore of general interest to address concerns about dye photodestruction and on-off emission state transitions typical of single molecule observations (see, Tinnefeld et al., Single Molecules, 1:215-223 (2000)).

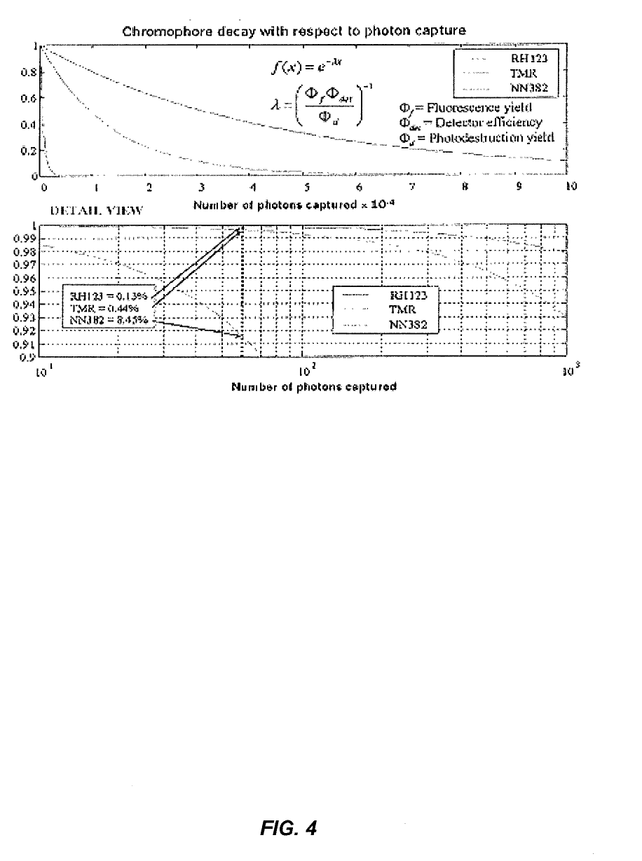

Photodegradation can limit the efficiency of single molecule detection if the dye “burns out” before it has emitted enough fluorescence photons to be detected. One of the better dyes is tetramethylrhodamine (TMR) having a photodestruction probability of 3.3E-07 per excitation event. Given a net optical collection and photon detection efficiency of 0.45%, and given that 60 photons are sufficient for detection (see, Tinnefeld et al., Single Molecules, 1:215-223, 2000)), it follows that a single TMR molecule must be excited 13,333 times (60/0.0045) to be detected. The probability that the molecule will photodegrade before 13,333 excitations is (1−exp(−13333×3.3E-07))=0.44%. This means that only 0.44% of molecules will escape detection due to photodegradation. This calculation is plotted in FIG. 4 for three different dyes, showing that TMR is in-between the performance of Rhodamine 123 (0.13% undetected) and NN382 (8.45% undetected). On-off fluorescence blinking behavior has been reported for the single dye molecules Cy-5 and JA242: both showed two “off” state components, one of 0.5 msec and the other around 5 msec (see, Tinnefeld et al., Single Molecules, 1:215-223 (2000)). The temporal aspect of blinking should not be a problem in our system because we acquire images for long periods (>20 msec) compared to the 5 msec “off” times, so that the moving path of most molecules is apparent in each image and across a series of images (movies). Because the quantum yield Φ_{f} is an average of the “on” and “off” states, the effects of blinking are implicit in the averaged calculations of FIG. 4, and individual molecules detected in the “on” state should actually be brighter than the average luminescence implied by the quantum yield.

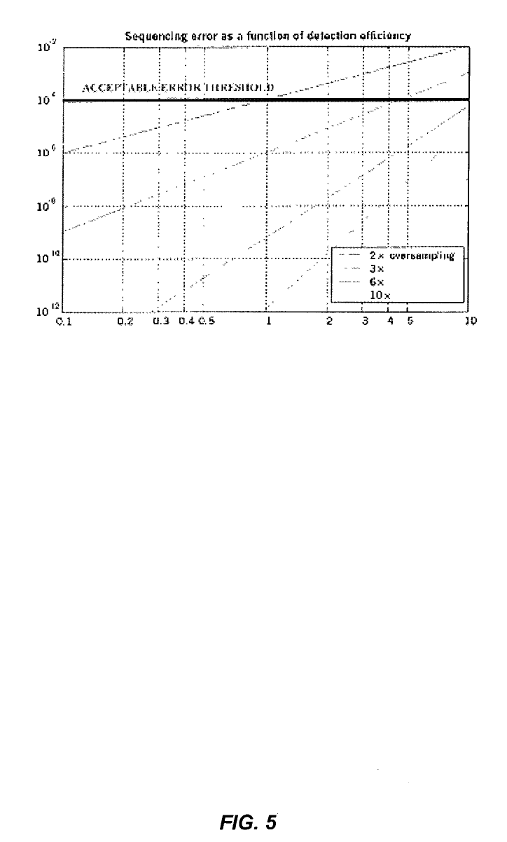

Error correction by oversampling. Since it is not possible to detect 100% of dye molecules, it is desirable to sequence a given DNA molecule (or entire genome) several times over to identify missing bases. FIG. 5 shows that the DNA sequencing error standard of 10⁻⁴ can be achieved by 6-fold oversampling given a detection efficiency of 90% and assuming that a base call is “real” if it appears in at least 2 of 6 reads. Most dyes to be detected with greater than 90% efficiency (FIG. 4). Oversampling is the standard means for error-correction in conventional DNA sequencing.

2.2 Activity of Naturally Occurring Polymerase for γ-dNTPs

As indicated by the following data, naturally occurring polymerases examined have relatively limited activity towards charge-switch nucleotides.

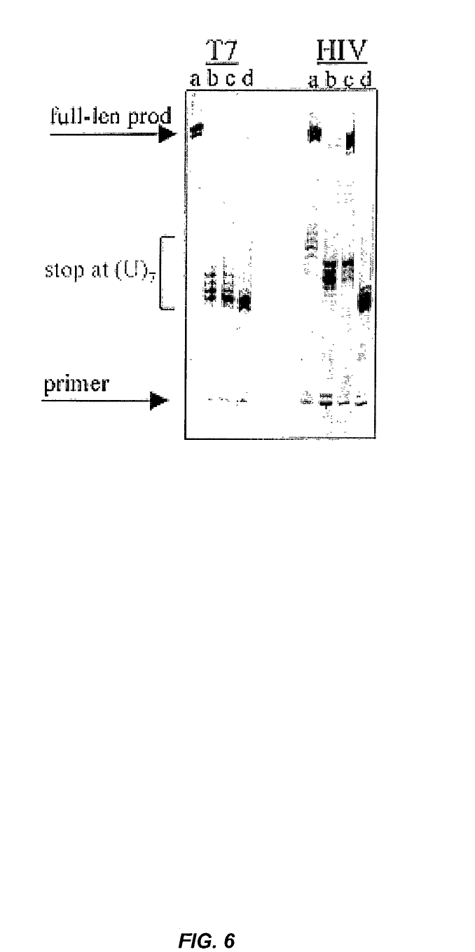

18 commercially-available polymerases were screened for the utilization of γ-dUTP-BodipyTR. HIV-1 RT utilized this substrate to produce full-length product after 30 min incubation, though it paused at a region of seven consecutive dUTP incorporation sites. In another experiment comparing incorporation of γ-dUTP labeled with either BodipyTR or fluorescein, HIV-1 RT incorporated the Bodipy substrate less efficiently than fluorescein, still pausing at a region of seven consecutive incorporation sites (FIG. 6). In the same experiment, T7 DNA polymerase barely incorporated the γ-dUTP analogs and it stopped at the consecutive incorporation sites. Positive controls showed that both enzymes synthesized full-length product with unlabeled dUTP (FIG. 6).

3. Preparation of Reagents for the Screen

3.1 Cloning And Expression of T7 And 429 Polymerase Genes

Cloning and Expression The polymerase genes were cloned into expression plasmids by 20-30 cycles of amplification from the respective phage genomes. A total of 16 clones were sequenced. Pfu DNA polymerase showed the greatest fidelity, giving 8 perfect clones out of 10, while the 6 clones amplified by Vent polymerase had 1-7 mutations each. The T7 polymerase was cloned with two intentional mutations built into the N-terminal PCR primer, D5A and E7A, which completely inactivate the 3′-5′ exonuclease (see, Patel et al., Biochemistry, 30:511-525 (1991)) and increase the apparent polymerization rate up to 9-fold (see, Tabor and Richardson, J Biol Chem, 264:6447-6458 (1989)). Four expression plasmids (Invitrogen) were used: pCR®T7/NT and /CT-TOPO which use the T7 RNA polymerase promoter and fuse 6× histidine tags to the N and C-terminus, respectively; pBAD/HisB which fuses a histidine tag to the N-terminus; and pBAD-HP which fuses “His-Patch Thioredoxin” (110 amino acids) to the N-terminus and a histidine tag to the C-terminus. The results were obtained for both enzymes using the pBAD vectors, inducing expression with arabinose and following protocols provided by Invitrogen.

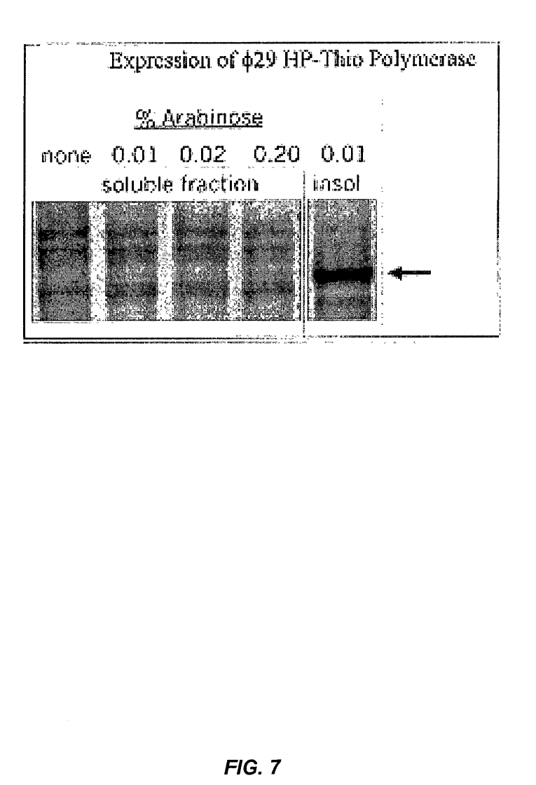

φ29 φ29 polymerase was strongly induced. Solubility was enhanced when φ29 polymerase was fused to the solubility-enhancing His-Patch Thioredoxin in a pBAD vector (Invitrogen) (FIG. 7).

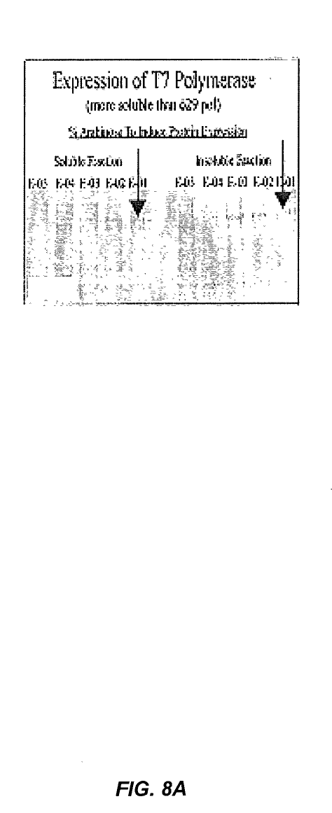

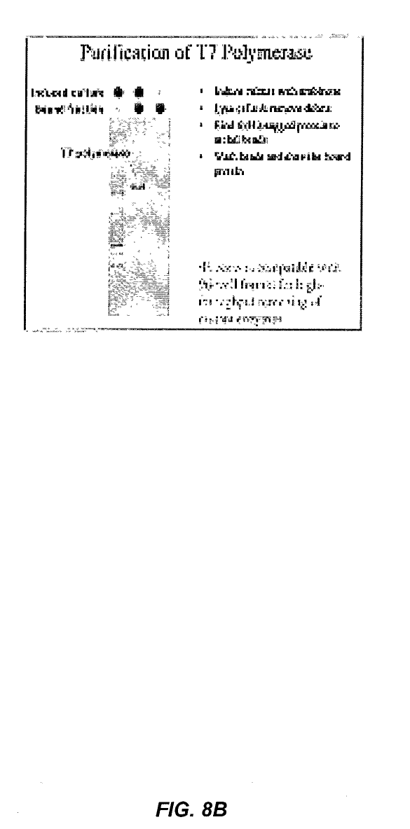

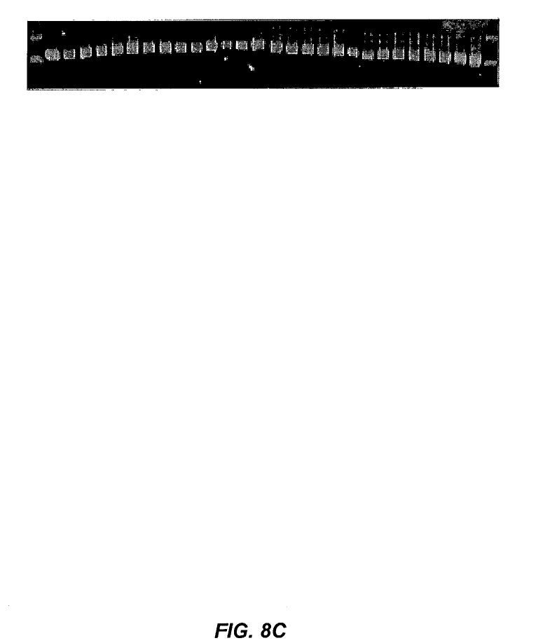

T7 Good expression of T7 DNA polymerase was obtained in the vector pBAD/HisB using 0.001% arabinose for 4 hours in E. coli TOP 10 cells (Invitrogen). Soluble protein was obtained in reasonable yield, approximating the amounts of the most abundant E. coli proteins, although a significant amount of the induced protein was insoluble (FIG. 8A).

3.2 Protein Purification in 96-Well Format

Purification Magnetic NTA agarose beads (Qiagen) were used to purify the soluble T7 polymerase from a single 1 ml culture according to the vendor's instructions. (FIG. 8B lane 3). In 96-well format, 1 ml cultures were grown in 2.4 mL-capacity square wells in a 96-well plate mounted on a tilted rotating drum at 32° C. Protein expression was induced by 0.002% arabinose for 3.5 hr and protein was purified as above using a magnet array for 96-well plates. Protein purified from 28 different cultures is shown in a Western blot to demonstrate the reproducibility of the method (FIG. 8C). The yield of purified protein was estimated at ˜3 μg protein per ml of induced culture as determined spectrophotometrically (E_{280 nm}=1.4E05 M⁻¹cm⁻¹, MW 83.5 kDa). Purity is estimated to be 98% by gel staining methods. Under polymerase assay conditions, there was no apparent endonuclease or exonuclease contamination. T7 polymerase is isolated in sufficient yield (2.2E13 molecules) and purity to run about 400 high-throughput screening assays (5E10 per assay) using a rapid 96-well procedure.

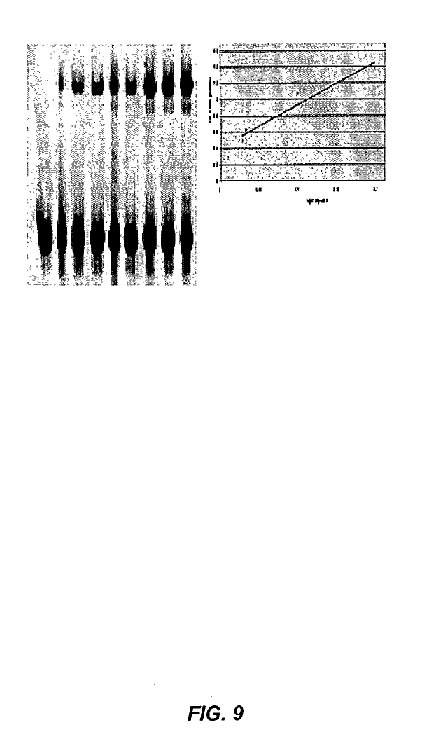

Steady-state kinetics. Kinetic measurements provide a way to characterize the improved polymerases. The Kₘ for dTTP was determined according to (Yang et al., Biochemistry, 38:8094-8101 (1999)), where the first base incorporated at the 3′-end of a primer is dTTP (in limiting concentrations), followed by run-off synthesis of 6 additional dGTP bases (in excess concentration); a Km of 13 uM was determined for dTTP from a Lineweaver-Burk plot (FIG. 9), which is close to the published value of 21 uM (Patel et al., Biochemistry, 30:511-525 (1991)).

3.3 Construction of T7 Pol- and Development of a Screening Assay For Detecting Polypeptides with Charge-Switch Polymerase Activity

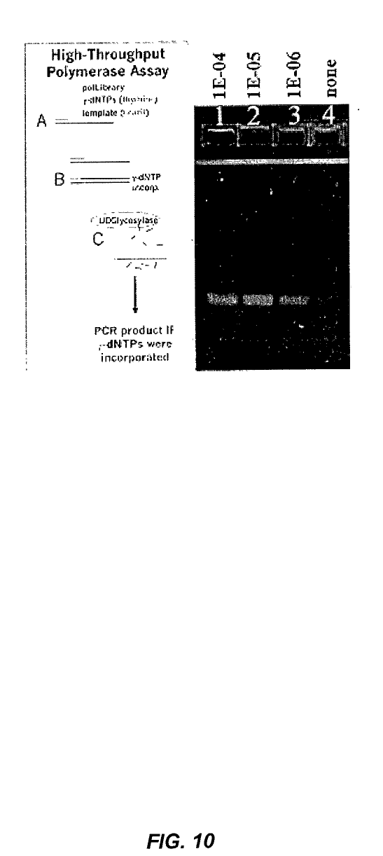

Assay. Uracil-DNA Glycosylase was used to degrade the template. A 100-nt synthetic oligonucleotide template (“U-DNA”) in which uracil is substituted for thymine was used. The primer is extended by polymerases using a dNTP mixture that includes thymine but not uracil; unused template is degraded by UDG; and surviving thymine-containing “T-DNA” is amplified by PCR (FIG. 10A). To demonstrate the assay, 5E10 molecules of primed U-DNA were mixed with 5E06, 5E06, 5E04 or 0 molecules of T-DNA. The samples were treated with UDG and amplified by 35 cycles of PCR (FIG. 10B). A small amount of amplicon was visible in a control sample without T-DNA (lane 4), but this was easily distinguished from the stronger bands obtained in samples containing T-DNA. FIG. 10 shows this assay is capable of million-fold discrimination, suitable for high-throughput screening of polymerase libraries.

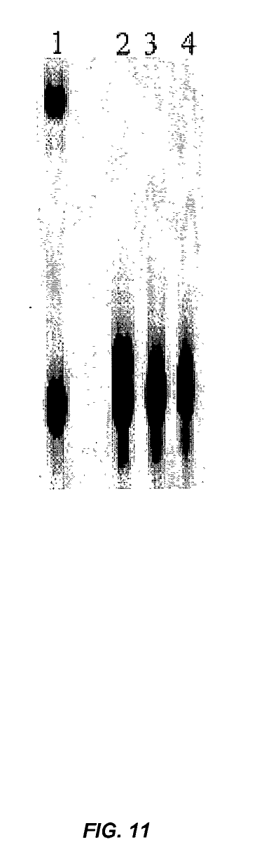

Construct a polymerase-defective mutant of T7 DNA polymerase exo-. A pol-mutant is used to provide a background of inactive mutants in a library containing pol+ enzymes; a pool deconvolution scheme is tested by isolating a pol+ clone using unlabeled dNTPs in the primer extension assay (above). Asp-654 chelates the active-site Mg++ in T7 polymerase (see, Doublie et al., Structure, 7:R31-R35 (1999)), so changing it to a non-acidic residue should inactivate the polymerization function. Stratagene's QuikChange kit was used to make a D654P mutation. The mutant protein was expressed and purified in the same yield as for the pol+ enzyme and was shown to have no polymerase activity, as desired (FIG. 11).

4. The Screen

Overview of Screen

Various charge-switched nucleotide structures (Table 2) are synthesized and evaluated for charge-switching behavior.

TABLE 2
Charge-Switch Nucleotides

| | | |
|---|---|---|
| BASE | LINKER | DYE |

Building Blocks

| | | |
|---|---|---|
| dATP | MQS (+1) | Alexa Fluor 488 (−2) |
| dCTP | BQS (+2) | Alexa Fluor 532 (−1) |
| dGTP | TQS (+3) | TAMRA (0) |
| dTTP | TetQS (+4) | Cy5 (−1) |
| MCA-dTTP | Pep (+2) | Bodipy TR (0) |
| BCA-dTTP | Pep (+3) | |

Set 1

| | | |
|---|---|---|
| A | BQS (+2) | TAMRA (0) |
| C | TQS (+3) | Alexa Fluor 532 (−1) |
| G | TQS (+3) | Cy5 (−1) |
| T | BQS (+2) | Bodipy TR (0) |

Set 2 (complement of Set 1)

| | | |
|---|---|---|
| A | TQS (+3) | Alexa Fluor 532 (−1) |
| C | BQS (+2) | TAMRA (0) |
| G | BQS (+2) | Bodiupy TR (0) |
| T | TQS (+3) | Cy5 (−1) |

Set 3 (Peptides of Set 1)

| | | |
|---|---|---|
| A | Pep (+2) | TAMRA (0) |
| C | Pep (+3) | Alexa Fluor 532 (−1) |
| G | Pep (+3) | Cy5 (−) |
| T | Pep (+2) | Bodipy TR (0) |

Nuc 1 (test TetQs (+4))

| | | |
|---|---|---|
| T | TetQS (+4) | Alexa Fluor 488 (−2) |

Nuc 2 (test MCA-dTTP)

| | | |
|---|---|---|
| MCA(−1)-dU | Pep (+2) | Bodipy TR (0) |

Nuc 3 (test BCA-dUTP)

| | | |
|---|---|---|
| BCA(−2)-dU | Pep (+3) | Bodipy TR (0) |

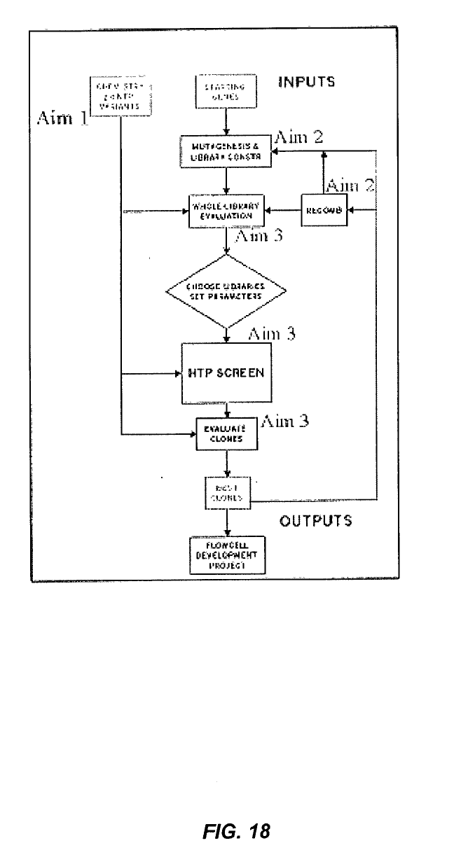

Next, DNA polymerases optimized to the various nucleotides are selected. Preferably, the polymerase has a synthesis rate of 10 nt/sec at γ-dNTP concentrations of 1-10 μM (lower concentrations conserve reagents and relax the microfluidics requirements). The breeding process is iterative (FIG. 18). Enzymes selected in the first cycle are recombined and/or further mutated for selection in subsequent cycles. Inputs are the T7 polymerase exo- and the various γ-dNTPs, such as those described in Example 2. The outputs are improved polymerases.

In one embodiment, the assay has the capability to screen an entire library of ˜1E06 variants in a single assay tube for activity with γ-dNTPs. TaqMan quantitative PCR, having a dynamic range of 1E05, should provide estimates of the number of clones in a given library that show activity at different γ-dNTP concentrations and incorporation times. The value of this capability cannot be overemphasized. Assay conditions and pool deconvolution dilution schemes can be optimized in advance. Mutation and recombination outcomes can be evaluated in different libraries with different classes of γ-dNTP.

4.1 Synthesis of Various Types of Charge-Switch Nucleotides

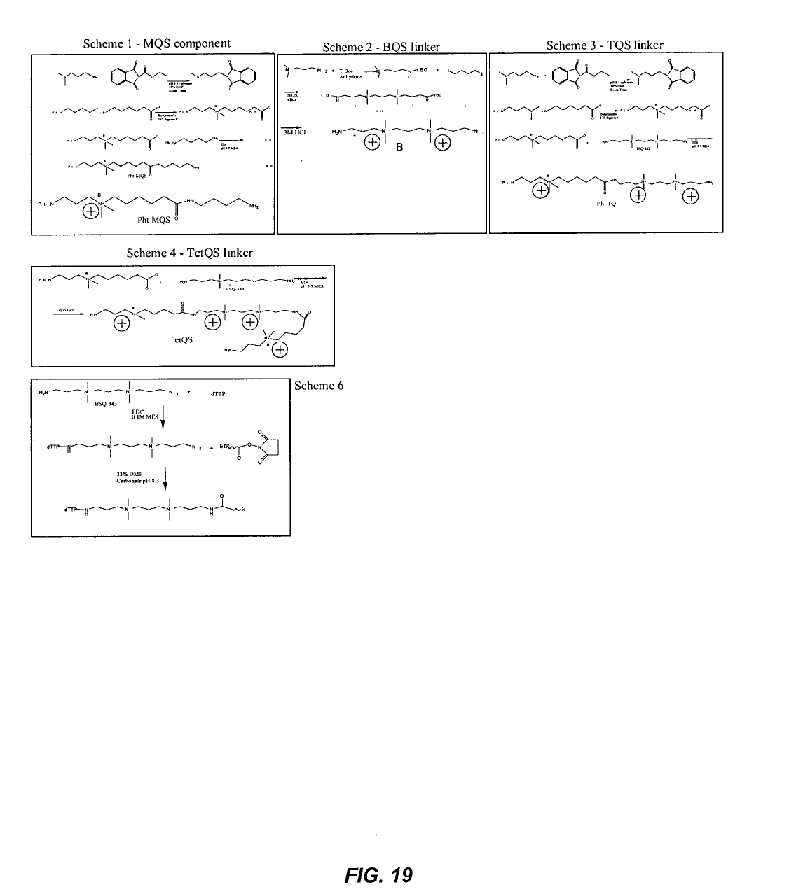

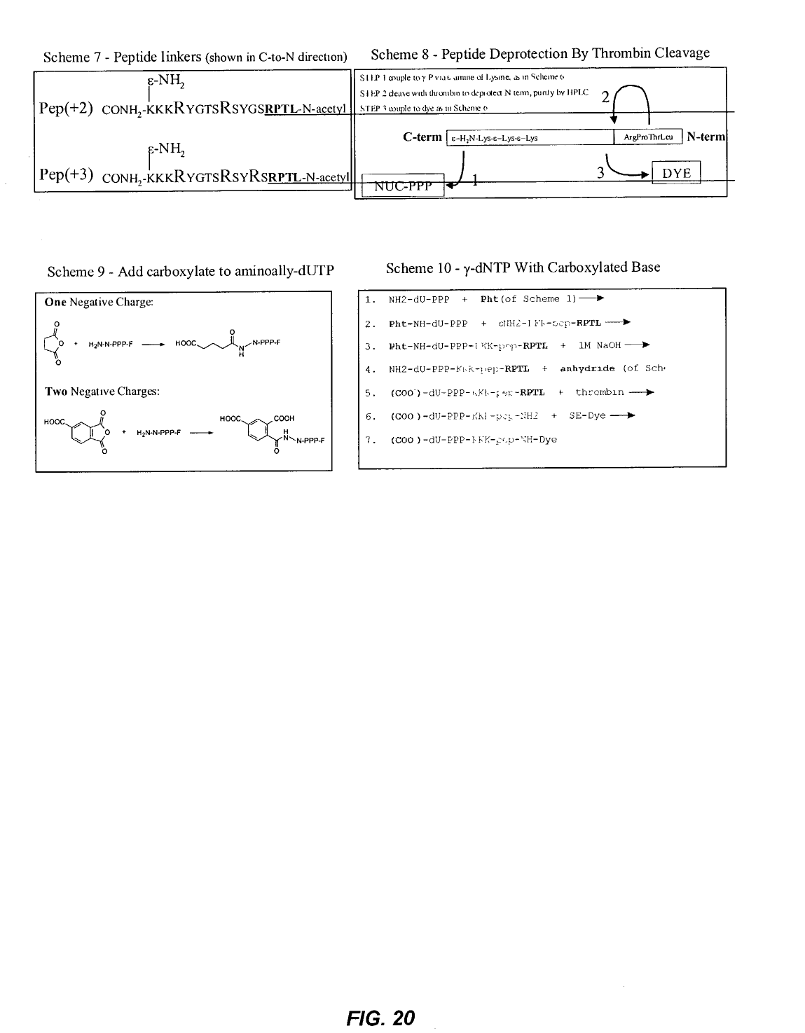

Various γ-dNTPs are synthesized and tested as polymerase substrates. Once an evolved polymerase is found to utilize a given γ-dNTP, then it is evaluated for charge-switching behavior by capillary electrophoresis. This section is organized around the building blocks and coupling chemistries that are used for synthesizing the nucleotides (Table 2, FIGS. 19-20).

4.1.1 Schemes 1-6 (FIG. 19)—Aliphatic Linkers; γ-Phosphate Conjugation

Scheme 1 The MQS(+) (monoquaternary salt) linker using a phthaliamide protecting group has been synthesized as shown. MQS is used as a reagent in Schemes 3 and 4.

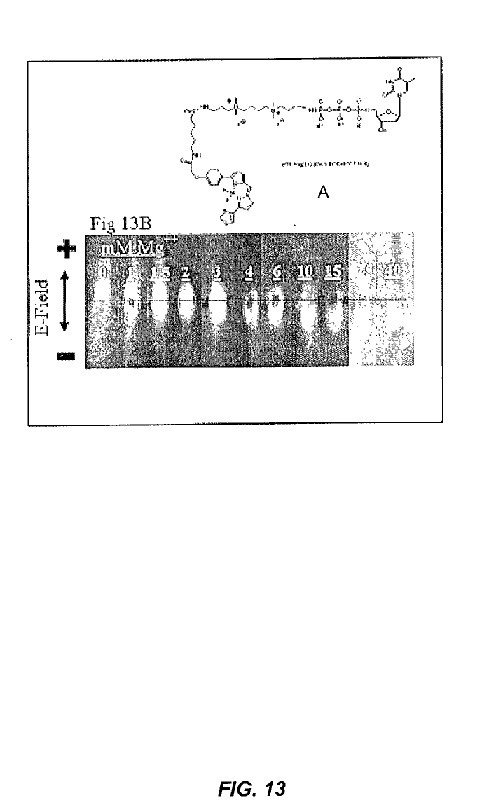

Scheme 2 The BQS(++) (bisquaternary salt) linker as shown has been synthesized and used it to synthesize several γ-dNTPs, including that of FIG. 13A.

Scheme 3 The TQS(+++) (triquaternary salt) linker by combining one MQS unit with one BQS unit has been synthesized using appropriate stoichiometry (Schemes 1,2). The phthaliamide protecting group is removed when necessary in 1M NaOH for 2h. dNTPs are stable in this condition.

Scheme 4 The TetQS(++++) (tetraquaternary salt) linker has been synthesized by combining two MQS units with one BQS unit as shown.

Scheme 5 Protection of the aminoally amino group of AA-dUTP is required in Scheme 10. The pthaliamide protecting group (see, Scheme 1) is used for this purpose.

Scheme 6 In this example, the BQS linker is coupled to dTTP. The product is purified by HPLC and reacted with the succinimide ester of BodipyTR.

4.1.2 Schemes 7-10 (FIG. 20)—Peptide Linkers: Carboxylate-Derivatized Nucleobase

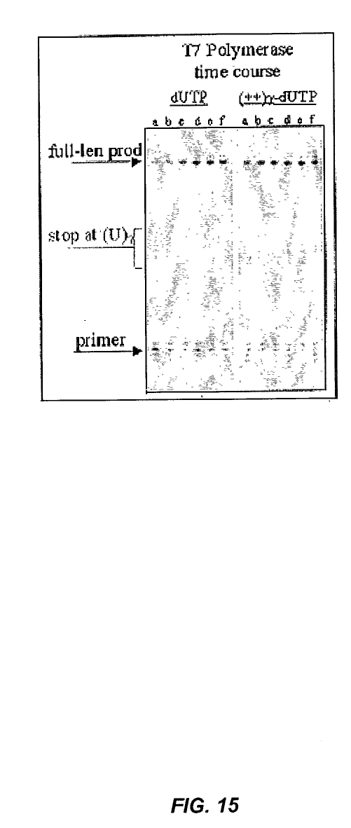

Scheme 7 Arginine residues carry a positive charge and are inert to the nucleotide coupling chemistry (Scheme 6). γ-dTTP-peptide(++)-BodipyTR and have shown that can be utilized by HIV-1 RT. The 3 lysines (KKK) are coupled through their ε-amines so that each residue provides 7 atoms to the linker. The three lysines together form a largely-aliphatic linker 21 atoms long, about the same as the BQS linker successfully utilized in a γ-dTTP by T7 polymerase (FIGS. 13A and 15). Both the C and N-termini of the peptide are permanently blocked by amidation or acylation. A reversible protecting group is required to achieve directional coupling. A protecting group, such as the sequence RPTL (C—N direction) which is cleaved very specifically by thrombin on the C-terminal side of the Arginine (Harris et al., Proc Nat Acad Sci USA, 97:7754-7759 (2000)), can be used.

Scheme 8 The peptides of Scheme 7 are coupled directionally to the γ-P of dNTPs as shown.

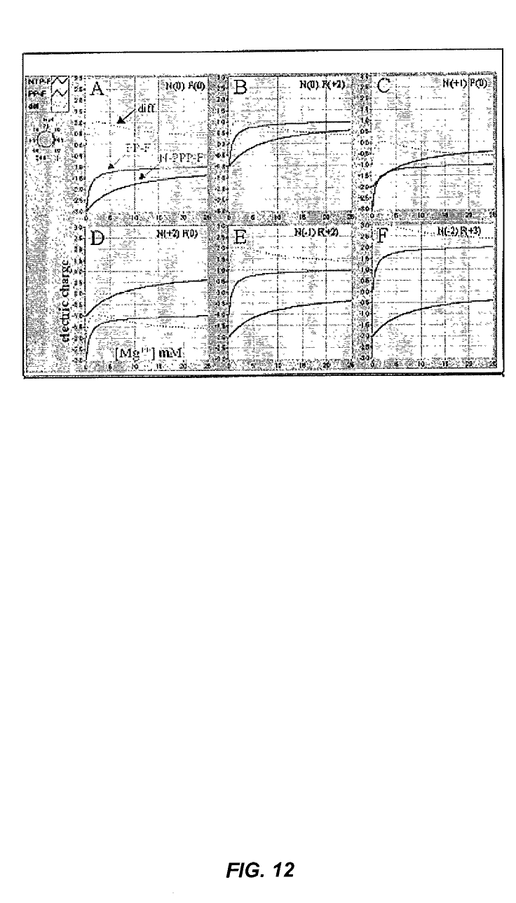

Scheme 9 The aminoallyl group of AA-dUTP is carboxylated with succinic anhydride (−1) or 1,2,4-benzenetricarboxylic anyhdride (−2). This provides negatively charged bases to test the high-magnitude charge-switch configurations of FIGS. 12E and 12F.

Scheme 10 Peptide linkers are used to synthesize the carboxylated γ-dUTPs mentioned in Scheme 9. These compounds are identified as Nuc1 and Nuc2 in Table 2 (MCA is “mono-carboxylic acid”; BCA is “bis-carboxboxylic acid”)

4.1.3 Specific Nucleotides To Synthesize (Table 2)

15 nucleotides listed in Table 2 were made (Set1, Set2, Set3, Nuc1, Nuc2, Nuc3) using the chemistry of Schemes 1-10.

4.2 Construction of a Mutant Polymerase Library

4.2.1 Mutagenesis by Error-Prone PCR

Error-prone PCR can be used to introduce random point mutations. A mutation frequency of 1-4 amino acid changes per protein is typical. While higher mutation rates can produce greater improvements (see, Daugherty et al, Proc Natl Acad Sci USA, 97:2029-2034 (2000)), the downside is that fewer clones retain activity and so there is a smaller pool from which to select improved variants. Kits such as Stratagene's GeneMorph™PCR Mutagenesis Kit employ a novel polymerase, Mutazyme™, that can be used to produce all possible transition and transversion mutations with minimal bias, and the mutation rate is controlled simply by the number of PCR cycles.

4.2.2 Site Saturation Mutagenesis

Having identified amino acid positions that improve activity in selected mutants, testing all amino acid substitutions at these sites can lead rapidly to even greater improvements. Site-saturation mutagenesis is useful because the single point mutations generated by PCR access only 5.7 amino acid substitutions on average, leaving untested the majority of possible substitutions (see, Miyazaki and Arnold, J Mol Evol, 49:716-720 (1999)). A published modification of Strategene's QuikChange site-directed mutagenesis protocol allows for simple and efficient library construction (see, Sawano and Miyawaki, Nucl Acids Res, 28:e78-e78 (2000)). Degenerate oligonucleotides targeted to multiple sites are used in a single-tube reaction with double-stranded plasmid as the template. Both mutants and recombinants between the different primers are generated in a single reaction. The QuikChange kit and the modified method (see, Sawano and Miyawaki, Nucl Acids Res, 28:e78-e78 (2000)) can be used for multisite mutagenesis.

4.3 Identification of Desired Clones with High-Throughput Screening

This section begins with a discussion of how clones are isolated from libraries, followed by more detailed descriptions of how whole libraries are characterized, of how high-throughput screening is conducted on the most promising libraries, and of how isolated clones are characterized.

4.3.1 Clone Isolation by Pool Deconvolution

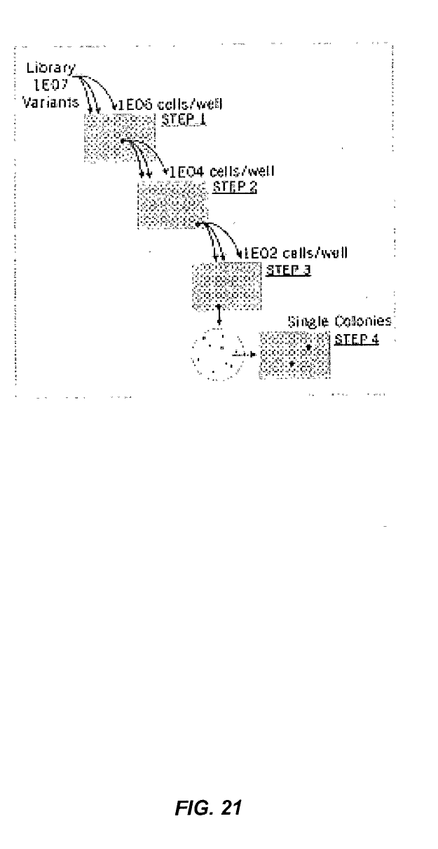

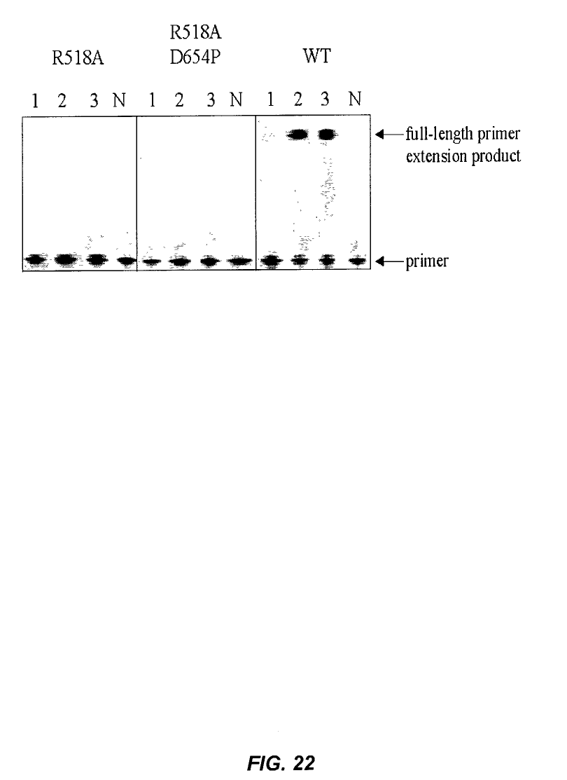

A geometric pool deconvolution scheme is used to isolate clones from bacterial libraries (FIG. 21). Positive pools are diluted into smaller pools and tested finally as individual clones. An average of 1.6 plates are required at each dilution step to capture every clone.

4.3.2 Whole-Library Characterization

Many more libraries can be generated than can be subjected to high-throughput screening for clone isolation. It is therefore of interest to characterize them as whole libraries with respect to enzyme kinetics to identify the most promising ones for screening. This also allows for the screening conditions to be optimized before starting the high-throughput screen. The number of clones that have activity at different γ-dNTP concentrations and reaction times are estimated by TaqMan quantitative PCR for each new library and γ-dNTP set. Whole-library characterization depends on the capability to perform quantitative PCR.

4.3.3 High-Throughput Screen

A flowchart of the screening process for isolating clones from the libraries by pool deconvolution is shown in FIG. 18. Histidine-tagged polymerase is expressed and purified from E. coli cultures in 96-well format using Qiagen Ni-NTA magnetic beads. A Qiagen turn-key robot is used to purify His-tagged proteins starting from bacterial cells and using the Qiagen reagent system. Purified protein is stored at ˜100 nM concentration with a 1000-fold molar excess of thioredoxin processivity factor (Sigma) in buffered 50% glycerol at −20° C. Protein is diluted 12-fold just before use to 8 nM in assay buffer (30 mM TrisCl pH 8, 10 mM MgCl₂, 1 mM DTT). Four μL of 8 nM polymerase (2E10 protein molecules) is transferred with a 96-tip pipetting machine (having 0.1 μL precision) into a plate preloaded with 1 μL of γ-dNTPs plus primed template DNA (2E10 DNA molecules, preannealed). The polymerase:DNA ratio is ˜1:1. Mixing is by pipetting up and down in the 96-tip machine. The incorporation reaction (5 μL) takes place in the tips during mixing, using reaction times as short as a few seconds (Section 3.2). A small 5 μL volume is used to conserve γ-dNTPs, but the volume are increased if necessary for successful pipetting.

The incorporation reaction is terminated by simultaneously transferring 2 μL of each sample to a plate pre-loaded with 8 μL per well of uracil-DNA glycosylase (UDG) master mix that contains a slight molar excess of EDTA (2.5 mM) over the Mg++ contributed from the polymerase cocktail (diluted conc 2 mM). The EDTA is compatible with UDG activity while quenching the polymerase reaction. The sample plate is incubated in a hot-bonnet thermal cycler at 44° C. for 1 h followed by 95° C. for 15 min to excise uracil from the template DNA strands and cleave at the resulting abasic sites. Five μL (4E09 template equivalents) of each sample is transferred simultaneously by the pipetting machine to a plate preloaded with 45 μL of TaqMan master mix for quantitative PCR amplification. Since the assay was initially set up with 1 polymerase protein per DNA template, amplification from 4E09 templates (most having been destroyed by UDG) provides up to 4E03 surviving product strands for every active polymerase in a sample of 1E06 variants. This is plenty of template for amplification; the amount of surviving template per sample increases geometrically 100-fold with each successive screening cycle such that individual clones can be isolated in a few cycles (FIG. 21).

4.3.4 Characterization of Isolated Clones

Kinetics Cloned polymerases obtained from the high-throughput screens are characterized in order to pick clones for additional recombination/mutation selection cycles. The Kₘ for each γ-dNTP are determined using the a single-base incorporation assay. All four γ-dNTPs are available and all 4 of a set are mixed together for the kinetic experiments.

Long read length Preferably, the polymerases capable of delivering long read lengths, thousands of bases, for DNA sequencing are used. To evaluate the ability of each enzyme to synthesize long DNA strands, a common polymerase assay (see, Satuma et al., J Mol Biol, 283:633-642 (1998)) that employs a primed M13 single-stranded DNA template is used. The distribution of product strand length is estimated by gel electrophoresis.

4.4 Development of Additional Methods for the Screen

4.4.1 High-Throughput Screen with M13 Template

M13mp18 phage are grown in an E. coli dut- ung-conditional mutant to incorporate uracil into the newly synthesized single-stranded phage DNA. The DNA are purified using a commercial kit (Qiagen) and the UDG assay is tried using the M13 template.

4.4.2 φ29 DNA Polymerase

φ29 polymerase mutant libraries are screened the same as for T7.

4.4.3 StEP Recombination

Sequenced mutations are efficiently recombined using the mutant multisite QuikChange (Stratagene) method discussed above (see, Sawano and Miyawaki, Nucl Acids Res, 28:e78-e78 (2000)). Uncharacterized mutations, however, are recombined using the staggered extension process (see, Zhao et al., Nature Biotechnology, 16:258-261 (1998) according to published guidelines (see, Volkov and Arnold, Meth Enzmol, 328:456-463 (2000)).

### Example 2

### Optimization of Charge-Switching Properties of Nucleotides: Variation of Ionic Composition of Medium and Charged Groups Added to the γ-Label or Nucleobase

This example illustrates various embodiments of charge-switch nucleotides.

The change in electric charge between an intact γ-dNTP—F and its cleavage product PPi-F is sensitive to the ionic composition of the medium and to charged groups on the γ-label and/or nucleobase.

Charge In the absence of Mg⁺⁺. The net electric charge on a dNTP, and hence its electrophoretic mobility, is governed by the base ring nitrogens and by the three phosphates (see, Saenger W, Principles of Nucleic Acid Structure, Springer-Verlag (1984); Frey et al., J Am Chem Soc, 94:9198-9204 (1972); Frey et al., J Am Chem Soc, 94:8898-8904 (1972)). At pH 7.5, the bases are largely uncharged (nitrogen pKs of 3-4 and 9.5-10); the primary ionization of each phosphate (pK˜2) contributes three full negative charges; and the secondary ionization specific to the γ-phosphate oxygen (pK 6.5; Frey et al., J Am Chem Soc, 94:8898-8904 (1972)) should contribute a time-averaged charge of −0.9 according to equilibrium calculations, so the total charge on a dNTP is (−3.9). Because the terminal oxygen is replaced by a label moiety “F” in a γ-dNTP—F, the secondary ionization is eliminated and the charge on a γ-dNTP—F is (−3.0), given that F is neutral. After cleavage from the nucleotide, the charge on the PPi-F is −2.9, about the same as before cleavage because, although it has one less phosphate than the γ-dNTP—F, it has gained a terminal phosphate oxygen of pK ˜6.5 (see, Frey et al., J Am Chem Soc, 94:8898-8904 (1972)). Thus, the net charge on a γ-dNTP—F is about the same as the net charge on the released PPi-γDye. This is not useful for electrosorting.

Charge In the presence of Mg⁺⁺. Since Mg⁺⁺ is required by polymerase, it is interesting to consider its effect on nucleotide charge. Mg⁺⁺ binds to phosphate groups in a variety of coordination isomers that rapidly equilibrate at 10³ to 10⁵ sece⁻¹ (see, Frey et al., J Am Chem Soc, 94:9198-9204 (1972)). Because Mg⁺⁺ contributes positive charge, it modulates the electrophoretic mobility of a nucleotide on a sub-millisec time scale to impart a net fractional charge on a time-averaged basis. This time scale is short relative to microfluidic flows in our system, so average charge can be used as a basis in this system. Mg⁺⁺ ions, like protons, bind more tightly to terminal phosphates than to “internal” phosphates (see, Frey et al., J Am Chem Soc, 94:8898-8904 (1972)), meaning that Mg⁺⁺ may impart more positive charge to PPi-F than to γ-dNTP—F. This effect could be modulated by substituting other metals (Mn⁺⁺) for Mg⁺⁺. If sufficiently large, this difference could be utilized to sort PPi-F from intact γ-dNTP—F in a microchannel system for DNA sequencing. This effect is quantitated below in discussing FIG. 12. T7 DNA polymerase is fully active at Mg⁺⁺ and Mn⁺⁺ concentrations as low as 1 mM (see, Tabor and Richardson, Proc Nat Acad Sci USA, 86:4076-4080 (1989)).

Charged Nucleobases. Charge switching can be enhanced by attaching positive or negative charged groups to the nucleobase (normally neutral at pH 7.5). When the base is incorporated into DNA, the charged group is separated from the PPi-F to enhance the “natural” Mg⁺⁺-dependent charge effect.

Polarity. In qualitative terms, there are 10 possible charge-switch modes that could be exploited for microchannel sorting (neg to less neg, neg to zero, zero to pos, etc.). The two “bipolar” modes (negative to positive, positive to negative) are preferred for electrosorting. In order to obtain a bipolar mode, it is necessary to “poise” the γ-dNTP with respect to charge so that the charge switch “passes through” neutral. This concept is illustrated in FIG. 12 which shows how Mg⁺⁺ ion affects the charge of generic γ-nucleotide (N—PPP—F) and cleavage product (PP—F). Six different charge configurations “N(b) F(g)” are shown, where b and g are the charge on the base or γ-label, respectively. The charged groups (having different pK's) were assumed to be primary or quaternary amines (+), or carboxylic acids (−) as detailed in the figure legend. With no added groups N(0) F(0) (Panel A), the maximum charge switch (Δq=+1) occurs at about 2 mM Mg⁺⁺, but the change is all in negative territory (−2.5 to −1.5). By adding a charge of (+2) to the γ-label (Panel B), the same switch magnitude is obtained (Δq=+1), except now it's shifted into bipolar mode where the γ-dNTP—F and PPi-F are oppositely charged (−0.5 to +0.5). Other configurations in FIG. 12 show how the charge switch magnitude can be further increased (to facilitate electrosorting) by adding various charges to the nucleobase and/or γ-label.

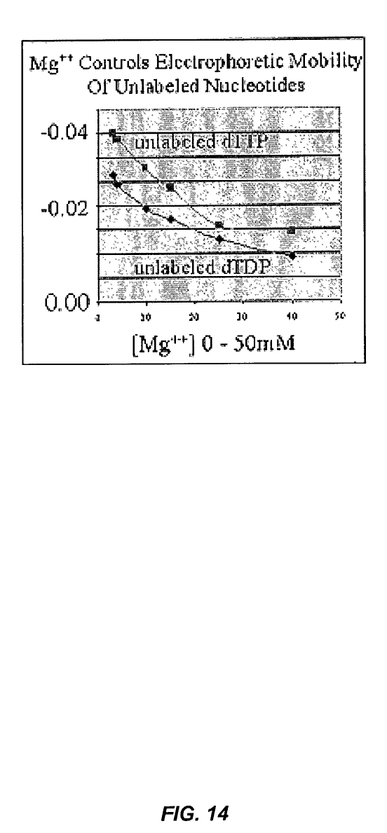

Electrophoresis Results. A γ-dNTP (FIG. 13A) with the charge configuration N(0) F(+2) was synthesized and its electrophoretic mobility examined in an agarose gel as a function of Mg⁺⁺ concentration (FIG. 13B). As expected (FIG. 12B), its mobility changed from negative to positive with increasing Mg⁺⁺, passing through zero at about 3 mM Mg⁺⁺. A direct comparison with the calculation (FIG. 12B) is not possible because, while the gels contained the indicated Mg⁺⁺ concentrations, the samples (20 μL) loaded in each lane contained 10 mM Mg⁺⁺. The importance of attaching a (+2) charge to the γ-label (FIG. 13A) with respect to obtaining a bipolar switch mode (neg to pos) is illustrated by a capillary electrophoresis experiment with unlabeled dTDP and dTTP (FIG. 14). Mg⁺⁺ imparted positive charge to both nucleotides, but both remained in negative territory. It is clear that additional positive charge can be added to the these nucleotides if one desires a negative to positive charge switch. This is what was done with the γ-dNTP of FIG. 13.

Charge-Switched γ-dTTP As A Polymerase Substrate. PPi-F was produced from the intact nucleotide N—PPP—F of FIG. 13A in a DNA synthesis reaction. The samples (containing 10 mM Mg⁺⁺, see ref to this in previous paragraph) were run on agarose gels containing different amounts of Mg⁺⁺, but no difference could be discerned in samples with or without HIV-1 RT. Other experiments established that HIV-1 RT was not cleaving enough nucleotide to be seen on an agarose gel.

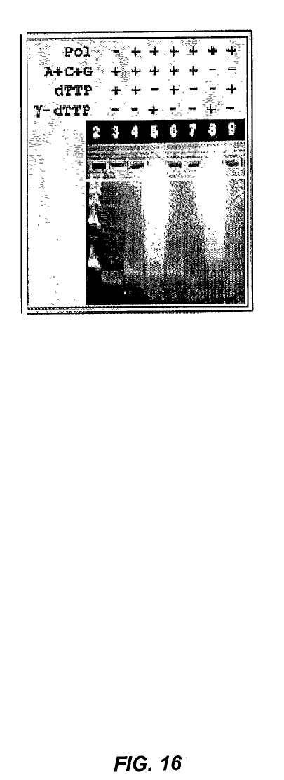

γ-dTTP is utilized by T7 as efficiently as unlabeled dTTP with a 50-mer oligonucleotide template (FIG. 15). This result was highly reproducible. To rule out the possibility of contamination, the γ-dTTP-BQS(++)-BodipyTR was analyzed by HPLC for unlabeled dTTP: none was found. Another experiment was done with a different template (the 100 mer used for the high-throughput polymerase assay) to try to detect dTTP contamination in other components of the reaction mix (FIG. 16): none was found. PPi-F is produced using T7 polymerase. The cleavage product is purified free from Mg⁺⁺.

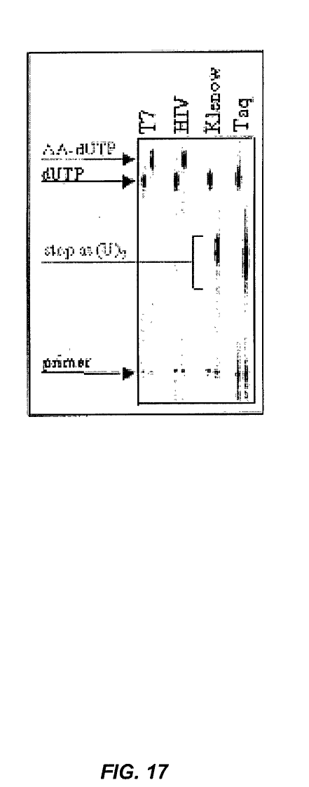

Charged Nucleobase as a Polymerase Substrate. Charge-switching can be enhanced by adding charged groups to the nucleobase (FIG. 12). Aminoallyl-dUTP was tested with 4 different polymerases. AA-dUTP should have a single (+) charge on the base at pH 7.5. T7 and HIV polymerases produced full-length product; Klenow and Taq polymerases stopped at the dUTP incorporation sites (FIG. 17).

### Example 3

### Cloning φ29 Polymerase into the pBAD/Myc-HisC Expression Vector

The φ29 DNA polymerase gene was PCR amplified from φ29 phage DNA using high-fidelity PfuTurbo polymerase in the buffer supplied with the enzyme (Stratagene). Amplification primers were a forward primer having a BspHI restriction enzyme site (5′-acggtctcatgaagcatatgccgag) and a reverse primer having a HindIII restriction enzyme site (5′-tcgttcaagctttgattgtgaatgtgtc). The φ29 polymerase amplicon was cut with BspHI and HindIII. The pBAD/Myc-HisC plasmid vector (Invitrogen) was cut with NcoI and HindIII. Both the amplicon and the vector were extracted with phenol and purified on Microcon PCR centrifugal filters (Millipore). The amplicon and vector were ligated together, transformed into E. coli TOP 10 (Invitrogen), and individual clones were sequenced to confirm their structure (SEQ. ID. NO: 37). In SEQ. ID. NO: 37 (5772 bp), the φ29 polymerase ORF is nucleotides 320-2044 and a C-Terminal fusion comprising a myc epitope tag and a 6× histidine tag is from nucleotides 2055-2116.

### Example 4

### φ29 Polymerase Expression and Purification

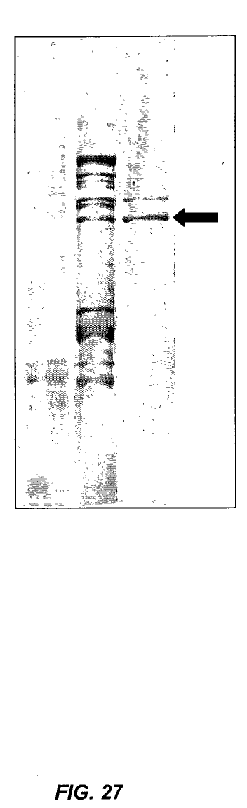

A log-phase culture of the clone SEQ. ID. NO: 37 was grown at 37° C. to a density of A600=0.5 in LB. Arabinose was added to 0.04% (w/v) and the culture was grown for 3.5 hr at 32° C. to allow for protein expression. Cells were harvested by centrifugation and stored at −80° C. until use. Frozen cells from 1 mL of culture were resuspended in 50 uL of lysis buffer #1 (50 mM NaH₂PO₄ pH 8.0, 300 mM NaCl, 10 mM imidazole, 0.05% Tween-20, 20% PEG 300), 0.5 μL of lysozyme (50 mg/mL) was added, the cells were frozen in liquid nitrogen, thawed and incubated on ice for 15 min, mixed with 150 μL of lysis buffer #2 (50 mM NaH₂PO₄ pH 8.0, 300 mM NaCl, 10 mM imidazole, 0.05% Tween-20, 1× Complete Protease Inhibitor Without EDTA and frozen in liquid nitrogen. The sample was thawed and mixed with 0.2 μL of DNAse 1 (5.6 mg/mL) and 1 μL of 1M MgCl₂ and incubated on ice for 10 min. Insoluble material was removed by centrifugation and the soluble His-tagged φ29 polymerase was purified with Ni—NTA magnetic beads following a procedure recommended by the vendor. Samples were analyzed by PAGE-SDS electrophoresis (FIG. 27).

### Example 5

### Strand Displacement Synthesis by φ29 Polymerase

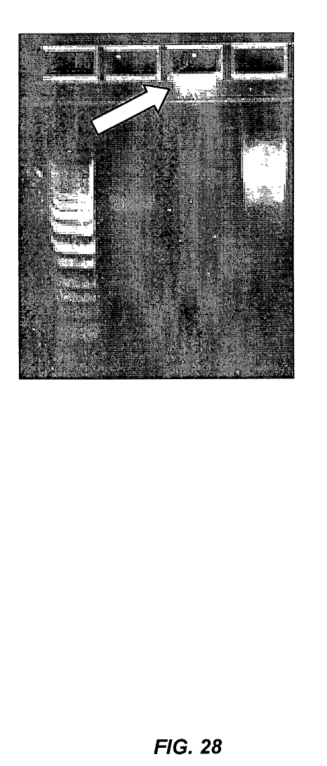

Purified C-Terminal His-tagged φ29 polymerase was tested for strand-displacement DNA synthesis using a primed M13 ssDNA template. Reaction mixtures contained M13 DNA (8 nM), primer (100 nM; 5′-gtaaaacgacggccagt), dNTPs (200 μM ea) in 50 mM TrisCl pH 7.8, 10 mM MgCl₂, 1 mM DTT. Samples were heated to 95° C. for 1 min, cooled, mixed with polymerase, incubated 1 hr at 37° C. SDS was added to 0.1% and the samples were heated at 65° C. for 10 min to remove any protein bound to the DNA. The samples were analyzed on an agarose gel (FIG. 28).

### Example 6

### φ29 exo- pol-double mutant N62D:K383A

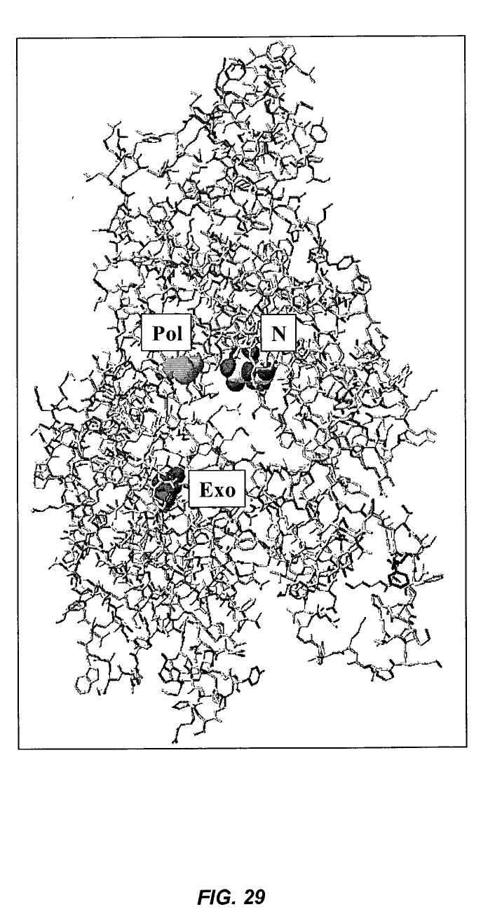

φ29 clone SEQ. ID. NO: 1 was mutated using the QuikChange site-directed mutagenesis kit. Primers for the N62D mutation (exo-) were 5′caagctgatctatatttccatgacctcaaatttgacggag and 5′-ctccgtcaaatttgaggtcatggaaatatagatcagcttg. Primers for the K383A mutation (pol-) were 5′-gagcgatcaagcaactagcagcactgatgttaaacagtctatac and 5′-gtatagactgtttaacatcagtgctgctagttgcttgatcgctc The N62D mutation was made first. A clone carrying the N62D mutation was then further mutated to K383A. The sequence of the double mutant is SEQ. ID. NO: 38. The locations of both mutations are indicated in a structural model of φ29 polymerase (FIG. 29).

### Example 7

### Screening Assay

A screening assay is used to test mutant libraries for the presence of polymerases capable of utilizing charge-switch nucleotides. In the version of the assay described here, a primed oligonucleotide template containing uracil is mixed with polymerase mutants in the presence of charge-switch nucleotides. The nucleotide mixture contains thymine bases, but no uracil bases. If an active polymerase is present, a new DNA strand containing thymine will be synthesized. The sample is then treated with uracil-DNA glycosylase (UDG) to degrade the uracil-containing template but not the thymine-containing product strand. A PCR reaction is then performed to detect surviving product strands.

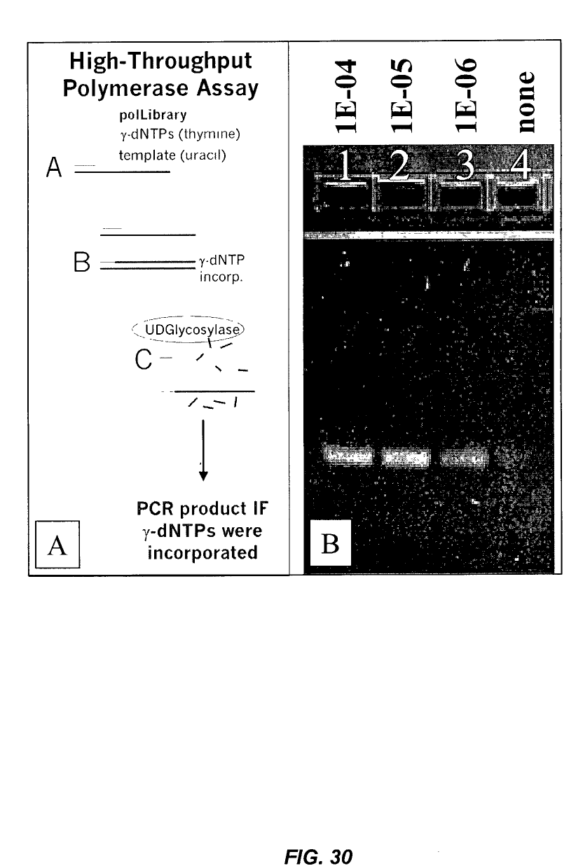

In this experiment (FIG. 30), thymine-containing strands were synthesized using non-charge-switch nucleotides. The thymine-containing DNA was mixed in different amounts with a fixed amount of uracil-containing template to determine the sensitivity of the assay. The template “U-DNA” is (5′acctutgacguggcguggctugtttcutattcutgcaucttaucgcccaccaucgaagauctcugagtutcaaauggaaauaac gggccaaccaccutga); the polymerase primer is (5′tcaaggtggttggcccgtt); the two PCR primers are (5′tcaaggtggttggcccgtt; same as the polymerase primer) and (5′acctttgacgtggcgtg). Double-stranded “T-DNA” was prepared in advance by incubating at 72° C. for 5 min the primed U-DNA with dNTPs containing dTTP and Taq polymerase. Test samples (10 μL) contained 5E10 molecules of primed U-DNA, plus 5E06, 5E05, 5E04 or 0 molecules of D-DNA (lanes 1-4, respectively, indicated by the ratio of D-DNA to U-DNA) in 50 mM TrisCl pH 9, 20 mM NaCl, UDG (100 u/ml; Epicentre). After incubating at 44° C. for 60 min, samples were heated at 95° C. to inactivate the UDG and to cleave abasic sites in the treated DNA. Two μL of each sample was diluted into a final volume of 10 μL containing 1× TaqGold Master Mix (Applera), 2.5 mM MgCl₂, 200 μM each dATP, dCTP, dGTP, dUTP, 1 μM each of the first and second PCR primer (above) and TaqGold polymerase (100 U/ml). PCR products were analyzed by agarose gel electrophoresis. UDG treatment can be supplemented with single-strand-specific nucleases to improve the assay sensitivity and specificity.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-36. Cancel

37. A purified mutant DNA polymerase having at least one mutation in a nucleotide γ-phosphate region, wherein said mutant DNA polymerase has increased activity for a γ-phosphate labeled nucleotide compared to a naturally occurring DNA polymerase, and wherein said mutant DNA polymerase is capable of synthesizing DNA at a rate of at least 1 nucleotide per second.

38. The purified mutant DNA polymerase of claim 37, wherein said increased activity for said γ-phosphate labeled nucleotide is increased by at least 2-fold.

39. The purified mutant DNA polymerase of claim 37, wherein said polymerase has decreased activity for a non-γ-phosphate labeled nucleotide.

40. The purified mutant DNA polymerase of claim 39, wherein said decreased activity for said non-γ-phosphate labeled nucleotide is about 2-fold to about 20-fold.

41. The purified mutant DNA polymerase of claim 37, wherein said mutant DNA polymerase has decreased exonuclease activity but retains strand displacement activity.

42. The purified mutant DNA polymerase of claim 37, wherein said mutant DNA polymerase has at least two mutations.

43. The purified mutant DNA polymerase of claim 37, wherein said mutant DNA polymerase has at least three mutations.

44. A purified mutant DNA polymerase having at least one mutation in a nucleotide γ-phosphate region, wherein said mutant DNA polymerase has increased activity for a γ-phosphate labeled nucleotide compared to a naturally occurring DNA polymerase, and wherein said mutant DNA polymerase is capable of synthesizing DNA at a rate of at least 10 nucleotides per second.

45. The purified mutant DNA polymerase of claim 44, wherein said increased activity for said γ-phosphate labeled nucleotide is increased by at least 2-fold.

46. The purified mutant DNA polymerase of claim 44, wherein said polymerase has decreased activity for a non-γ-phosphate labeled nucleotide.

47. The purified mutant DNA polymerase of claim 46, wherein said decreased activity for said non-γ-phosphate labeled nucleotide is about 2-fold to about 20-fold.

48. The purified mutant DNA polymerase of claim 44, wherein said mutant DNA polymerase has decreased exonuclease activity but retains strand displacement activity.

49. The purified mutant DNA polymerase of claim 44, wherein said mutant DNA polymerase has at least two mutations.

50. The purified mutant DNA polymerase of claim 44, wherein said mutant DNA polymerase has at least three mutations.

51. A purified mutant DNA polymerase having at least one mutation in a nucleotide γ-phosphate region, wherein said mutant DNA polymerase has increased activity for a nucleotide coupled to a detectable moiety at a γ-phosphate compared to a naturally occurring DNA polymerase, and wherein said mutation is found in regions of a nucleotide binding pocket of said DNA polymerase, wherein said pocket interacts with said detectable moiety of the nucleotide.

52. The purified mutant DNA polymerase of claim 51, wherein said detectable moiety is selected from the group consisting of PPi-Dye, PP—F, P-Dye and P—F.

53. The purified mutant DNA polymerase of claim 51, wherein said detectable moiety is a phosphate detectable moiety that is cleaved from γ-labeled dNTPs.

54. The purified mutant DNA polymerase of claim 51, wherein said increased activity for a nucleotide coupled to a detectable moiety at a γ-phosphate is increased by at least 2-fold.

55. The purified mutant DNA polymerase of claim 51, wherein said polymerase has decreased activity for a non-γ-phosphate labeled nucleotide.

56. The purified mutant DNA polymerase of claim 55, wherein said decreased activity for said non-γ-phosphate labeled nucleotide is about 2-fold to about 20-fold.


SEQUENCE LISTING (52 sequences)

SEQ ID NO: 1 - DNA, 1725 bp, Bacteriophage phi-29
  CDS (1)..(1725): native wild-type phi29 DNA polymerase
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaagctctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagtcgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagcaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaa

SEQ ID NO: 2 - protein, 575 aa, Bacteriophage phi-29 [sequence omitted]

SEQ ID NO: 3 - protein, 575 aa, Artificial Sequence
  Description of Artificial Sequencephi29 polymerase with K383A mutation
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAALMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 4 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTPLFKDFIDKWTVIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 5 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKWGLDRMTAGSDSLKGFKDIITTKKFKKVFPTLSLGLDKEVRYAYRGGFTWLNDRFKEKEIGEGMVFDVNSLYVAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 6 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYSAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 7 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMSEVDGKLVEGSPDDYTDIKFSVKCAGMTDKIKKEVTFENFKVGFSRKMKPKPVQVPGGVVLVDDTFTIK

SEQ ID NO: 8 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIITVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDTIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 9 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTNKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 10 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVASLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 11 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDMNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESLFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 12 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGFFKDFISKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 13 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGKDRMTAGSDSLKGFKDIITTKKFKKVFPTLSLGLDKEVRYAYRGGFTWLNDRFKEKEIGEGMVFVVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 14 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMGFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFILKWTYIKTTSEGAIKQLAKLMCNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 15 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIYKWWYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 16 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKPVDGKLVEGSPDDYTDIKFSVKCAGMTDKIKKEVTFENFKVGFSRKMKPKPVQVPGGVVLVDDTFTIK

SEQ ID NO: 17 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIMDIYMKEVDGKLVEGSPDDYTDIKFSVKCAGMTDKIKKEVTFENFKVGFSRKMKPKPVQVPGGVVLVDDTFTIK

SEQ ID NO: 18 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKTMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 19 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMWFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 20 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMNFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQNIYMKEVDGKLVEGSPDDYTDIKFSVKCAGMTDKIKKEVTFENFKVGFSRKMKPKPVQVPGGVVLVDDTFTIK

SEQ ID NO: 21 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMPFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDPIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 22 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMEFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFDDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 23 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQFAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 24 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKISTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYVAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 25 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGNIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMLEVDGKLVEGSPDDYTDIKFSVKCAGMTDKIKKEVTFENFKVGFSRKMKPKPVQVPGGVVLVDDTFTIK

SEQ ID NO: 26 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQVKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 27 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVDDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 28 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLRKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 29 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKMHTVIYDSLKKLPFPVKKIAKDFKLTVLKGDIDYHKERPVGYKITPEEYAYIKNDIQIIAEALLMQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSESAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMMEVDGKLVEGSPDDYTDIKFSVKCAGMTDKIKKEVTFENFKVGFSRKMKPKPVQVPGGVVLVDDTFTIK

SEQ ID NO: 30 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLCSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIFDIYMKEVDGKLVEGSPDDYTDIKFSVKCAGMTDKIKKEVTFENFKVGFSRKMKPKPVQVPGGVVLVDDTFTIK

SEQ ID NO: 31 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYFKEVDGKLVEGSPDDYTDIKFSVKCAGMTDKIKKEVTFENFKVGFSRKMKPKPVQVPGGVVLVDDTFTIK

SEQ ID NO: 32 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSNYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKMFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMDEVDGKLVEGSPDDYTDIKFSVKCAGMTDKIKKEVTFENFKVGFSRKMKPKPVQVPGGVVLVDDTFTIK

SEQ ID NO: 33 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFIVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 34 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLITFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTCIKTTSEGAIKQLAKLYLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAMESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 35 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNGLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFNDFIDWWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWMHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 36 - protein, 575 aa, Artificial Sequence
  Description of Artificial Sequencenucleotide gamma-phosphate interaction region mutant phi29 DNA polymerase
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQIAKQMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 37 - DNA, 5772 bp, Artificial Sequence
  Description of Artificial Sequencephi29 polymerase in pBAD/Myc-HisC vector
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaattaaccatgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaagctctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagtcgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagcaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaagcttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 38 - DNA, 5772 bp, Artificial Sequence
  Description of Artificial Sequencephi29 exo- pol-double mutant N62DK383A in pBAD/Myc-HisC vector
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaattaaccatgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccatgacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaagctctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagtcgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagcaactacaggtttgtttaaagattttatagataaatggacgtacatcaagacgacatcagaaggagcgatcaagcaactagcagcactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaagcttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 39 - protein, 17 aa, Artificial Sequence
  Description of Artificial Sequencepeptide linker Pep(+2)
XTLRSGYSRSTGYRKKK

SEQ ID NO: 40 - protein, 17 aa, Artificial Sequence
  Description of Artificial Sequencepeptide linker Pep(+3)
XTPRSRYSRSTGYRKKK

SEQ ID NO: 41 - protein, 4 aa, Artificial Sequence
  Description of Artificial SequenceN-terminal protecting group
LTPR

SEQ ID NO: 42 - protein, 6 aa, Artificial Sequence
  Description of Artificial Sequence6x histidine tag
HHHHHH

SEQ ID NO: 43 - DNA, 17 bp, Artificial Sequence
  Description of Artificial Sequenceprimer
gtaaaacgacggccagt

SEQ ID NO: 44 - DNA, 40 bp, Artificial Sequence
  Description of Artificial Sequenceprimer for N62D mutation (exo-)
caagctgatctatatttccatgacctcaaatttgacggag

SEQ ID NO: 45 - DNA, 40 bp, Artificial Sequence
  Description of Artificial Sequenceprimer for N62D mutation (exo-)
ctccgtcaaatttgaggtcatggaaatatagatcagcttg

SEQ ID NO: 46 - DNA, 44 bp, Artificial Sequence
  Description of Artificial Sequenceprimer for K383A mutation (pol-)
gagcgatcaagcaactagcagcactgatgttaaacagtctatac

SEQ ID NO: 47 - DNA, 44 bp, Artificial Sequence
  Description of Artificial Sequenceprimer for K383A mutation (pol-)
gtatagactgtttaacatcagtgctgctagttgcttgatcgctc

SEQ ID NO: 48 - DNA, 100 bp, Artificial Sequence
  Description of Combined DNA/RNA Moleculetemplate “U-DNA”
acctutgacguggcguggctugtttcutattcutgcaucttaucgcccaccaucgaagauctcugagtutcaaauggaaauaacgggccaaccaccutga

SEQ ID NO: 49 - DNA, 19 bp, Artificial Sequence
  Description of Artificial Sequencepolymerase primer, PCR primer
tcaaggtggttggcccgtt

SEQ ID NO: 50 - DNA, 17 bp, Artificial Sequence
  Description of Artificial Sequencesecond PCR primer
acctttgacgtggcgtg

SEQ ID NO: 51 - DNA, 26 bp, Artificial Sequence
  Description of Artificial Sequenceforward amplification primer having BspHI site
acggtctcatgaacgcatatgccgag

SEQ ID NO: 52 - DNA, 28 bp, Artificial Sequence
  Description of Artificial Sequencereverse amplification primer having HindIII site
tcgttcaagctttgattgtgaatgtgtc